(19) United States
(12) Patent Application Publication
(10) Pub. No.: US 2021/0164015 A1
(43) Pub. Date: Jun. 3, 2021

(54) ANTIMICROBIAL SUSCEPTIBILITY TESTING USING MICRODROPLETS

(71) Applicant: Becton, Dickinson and Company, Franklin Lakes, NJ (US)

(72) Inventors: Kristin Weidemaier, Raliegh, NC (US); David Sebba, Cary, NC (US); Rajendra Bhat, Cary, NC (US); Meghan Wolfgang, Pittsboro, NC (US); Pauline Bell, Wake Forest, NC (US)

(21) Appl. No.: 17/171,629

(22) Filed: Feb. 9, 2021

Related U.S. Application Data
(63) Continuation of application No. PCT/US2019/046478, filed on Aug. 14, 2019.
(60) Provisional application No. 62/719,290, filed on Aug. 17, 2018.

(51) Int. Cl. C12Q 1/18

(52) U.S. Cl. CPC C12Q 1/18

(57) ABSTRACT

Provided herein are compositions, methods, systems and/or kits for measuring microbial viability in a sample. Certain embodiments of the present disclosure are related to detection tests comprising compositions, methods, systems and/or kits for measuring an antimicrobial minimum inhibitory concentration and for measuring microbial susceptibility to the antimicrobial. Certain embodiments of the present disclosure are related to detection tests comprising compositions, methods, systems and/or kits for assessing microbial proliferation in a sample.

### CROSS REFERENCE TO RELATED APPLICATION

This application is a continuation of PCT Application No. PCT/US2019/046478, filed Aug. 14, 2019, which claims the benefit of U.S. Provisional Application No. 62/719,290, filed Aug. 17, 2018, each of which is hereby incorporated by reference in its entirety.

### BACKGROUND

### Field

The present disclosure is generally related to detection tests comprising compositions, methods, systems and/or kits for determining susceptibility of microorganisms in a sample to antibiotics. Certain embodiments of the present disclosure are related to detection tests comprising compositions, methods, systems and/or kits for measuring an antimicrobial minimum inhibitory concentration.

### Description of the Related Art

Microbial infection affects millions of people annually, with millions of fatalities per year. Rigorous diagnosis of pathogen type often requires days to obtain, even in state-of-the art clinical microbiology laboratories. Moreover, patients who initially receive incorrect therapies exhibit a lower survival rate than those who are treated with optimal therapy from early in the course of the disease. The rapidity of pathogen diagnosis in a patient with a microbial infection can have important prognostic ramifications. The current methods for detecting microbial infection in blood is to culture the blood in a hospital or commercial clinical microbiology laboratory. Liquid cultures can permit detection of the existence of some type of growing organism in the fluid within 4 to 30 hours. This assay is not quantitative and without knowledge of the type of pathogen and their specific antibiotic sensitivities, only wide-spectrum antibiotics can be administered at this time, which are suboptimal at best. To identify the specific type of pathogen, and to carry out sensitivity testing to determine their responses to various potential antibiotic therapies, the pathogens growing in liquid medium must then be transferred to other growth media (e.g., agar plates). The total time for full diagnosis and sensitivity testing is commonly 3-7 days and empiric antibiotic treatment based on clinical symptoms is started well before the results of the antibiotic sensitivity are obtained, often within 1-3 hours after blood cultures are first drawn from the patient.

Many patients with microbial infection exhibit a rapid decline within the early hours of infection. Thus, rapid and reliable diagnostic and treatment methods are essential for effective patient care. Unfortunately, current antimicrobial susceptibility testing techniques generally require a prior isolation of the microorganism by culture (e.g., about 12 to about 48 hours), followed by a process that requires another about 6 to about 24 hours. For example, a confirmed diagnosis as to the type of infection traditionally requires microbiological analysis involving inoculation of blood cultures, incubation for 16-24 hours, plating the causative microorganism on solid media, another incubation period, and final identification 1-2 days later. Even with immediate and aggressive treatment, some patients develop multiple organ dysfunction syndrome and eventually death.

Every hour lost before a correct treatment is administered can make a crucial difference in patient outcome. Consequently, it is important for physicians to rapidly determine whether an infection is present, and if so, which antimicrobial would be effective for the treatment.

### SUMMARY

Described herein are compositions, methods, systems and/or kits for measuring microbial viability in a sample.

Some embodiments provided herein relate to a method of assessing microbial proliferation in a sample. In some embodiments, the method includes providing a sample including microbes, separating the sample including microbes into one or more portions of sample including microbes, forming one or more populations of microdroplets encapsulating microbes from the sample, contacting the one or more portions of sample with an antimicrobial either before or after forming one or more populations of microdroplets, and measuring microbial viability of microbes encapsulated within microdroplets thereby determining susceptibility of the microbes to the antimicrobial. In some embodiments, the one or more populations of microdroplets are formed before or after separating the sample into one or more portions. In some embodiments, each of the one or more portions of the sample is contacted with a different concentration of an antimicrobial. In some embodiments, measuring microbial viability includes obtaining a measure of microbial viability from a discrete subset of microdroplets from a first population of microdroplets from a first portion of the sample measured at a first time point, and obtaining a measure of microbial viability from a discrete subset of microdroplets from a second population of microdroplets from the first portion of the sample measured at a second time point. In some embodiments, measuring microbial viability further includes comparing the measure of microbial viability from a discrete subset of microdroplets measured at the first time point to the measure of microbial viability from a discrete subset of microdroplets measured at the second time point for a plurality of subsets of microdroplets measured at the first and second time points. In some embodiments, measuring further includes obtaining a measure of microbial viability from a discrete subset of microdroplets in an additional population of microdroplets from the first portion of the sample measured at an additional time point. In some embodiments, an average of the measure of microbial viability from a plurality of discrete subsets of microdroplets measured at the first time point is compared to an average of the measure of microbial viability from a plurality of discrete subsets of microdroplets measured at the second time point. In some embodiments, the measurements of microbial viability obtained at the first and second time points are not assigned to discrete subsets of microdroplets. In some embodiments, one or more discrete subsets of microdroplets from the first population are not in the second population, and one or more discrete subsets of microdroplets from the second population are not in the first population. In some embodiments, the populations of microdroplets are incubated for a period of any one or more of 0 hr, 0.1 hr, 0.2 hr, 0.5 hr, 1 hr, 2 hr, 3 hr, 4 hr, 5 hr, 6 hr, 7 hr, 8 hr, 9 hr, 10 hr, 11 hr, 12 hr, 15 hr, 18 hr, 21 hr, or 24 prior to measuring microbial viability. In some embodiments, the microdroplets are formed within 1 second, 30 seconds, 1 minute, 15 minutes, 30 minutes, 1 hour, or 2 hours of contacting the one or more portions of samples with the antimicrobial. In some embodiments, measuring microbial viability further includes obtaining a measure of microbial viability from discrete subsets of microdroplets in a first population of microdroplets from a first portion of the sample measured at a first time point, and obtaining a measure of microbial viability from discrete subsets of microdroplets in a second population of microdroplets from the first portion of the sample measured at a second time point. In some embodiments, measuring further includes assigning measurements obtained at the first and second time points to discrete subsets of microdroplets, wherein at least some of the discrete subsets of microdroplets in the first population are the same discrete subsets of microdroplets in the second population such that the measurement of microbial viability obtained for a discrete subset of microdroplets at the first time point can be compared to the measurement of microbial viability obtained for that same discrete subset of microdroplets obtained at the second time point.

In some embodiments, measuring microbial viability further includes comparing the measurement of microbial viability obtained for a discrete subset of microdroplets at the first time point to the measurement of microbial viability obtained for that same discrete subset of microdroplets obtained at the second time point. In some embodiments, at least one discrete subset of microdroplets in the first population is not in the second population, and at least one discrete subset of microdroplets in the second population is not in the first population. In some embodiments, measuring further includes obtaining a measure of microbial viability from a discrete subset of microdroplets in an additional population of microdroplets measured at an additional time point. In some embodiments, measuring microbial viability includes obtaining a measure of microbial viability from a discrete subset of microdroplets in a first population of microdroplets from a first portion of the sample measured at a first time point, and obtaining a measure of microbial viability from a discrete subset of microdroplets in a second population of microdroplets from the first portion of the sample measured at a second time point. In some embodiments, the measure of microbial viability is whether an indicator of microbial viability exceeds a preset threshold. In some embodiments, the composite of the measure of microbial viability is the percentage of the plurality of discrete subsets of microdroplets measured at a time point that exceeds the threshold. In some embodiments, the preset threshold is exceeded when an indicator reaches a determined measure of microbial viability. In some embodiments, a composite of the measure of microbial viability from a plurality of discrete subsets of microdroplets measured at the first time point is compared to a composite of the measure of microbial viability from a plurality of discrete subsets of microdroplets measured at the second time point. In some embodiments, measuring microbial viability further includes comparing the measure of microbial viability from a discrete subset of microdroplets obtained at the first time point to the measure of microbial viability from a discrete subset of microdroplets obtained at the second time point for a plurality of subsets of microdroplets measured at the first and second time points. In some embodiments, the measurements of microbial viability obtained at the first and second time points are not assigned to discrete subsets of microdroplets. In some embodiments, the measuring microbial viability further includes comparing the measure of microbial viability obtained for a discrete subset of microdroplets at the first time point to the measure of microbial viability obtained for that same discrete subset of microdroplets obtained at the second time point, for a plurality of subsets of microdroplets. In some embodiments, one or more discrete subsets of microdroplets from the first population are not in the second population, and one or more discrete subsets of microdroplets from the second population are not in the first population. In some embodiments, measuring further includes obtaining a measure of microbial viability from a discrete subset of microdroplets in an additional population of microdroplets measured at an additional time point if an indicator of microbial viability exceeds the preset threshold. In some embodiments, the method further includes incubating the one or more portions of samples contacted with antimicrobial for different periods of time prior to forming the one or more populations of microdroplets, whereby microdroplets are formed from each of the one or more portions of samples at different time points. In some embodiments, the one or more portions of samples are incubated for a time period sufficient to monitor microbial viability. In some embodiments, the one or more portions of samples are incubated for a time period sufficient to allow microbial quorum sensing. In some embodiments, the one or more portions of samples are incubated over a period of any one or more of 0 hr, 0.1 hr, 0.2 hr, 0.5 hr, 1 hr, 2 hr, 3 hr, 4 hr, 5 hr, 6 hr, 7 hr, 8 hr, 9 hr, 10 hr, 11 hr, 12 hr, 15 hr, 18 hr, 21 hr, or 24 hr prior to forming microdroplets. In some embodiments, susceptibility of a microorganism to an antibiotic is determined by measuring viability of microorganisms in the presence of difference concentrations of an antibiotic.

In some embodiments, measuring microbial viability in droplets is performed using a technology that affects viability of the microorganism, including determination of bacterial concentration by genetic analysis, including qPCR or fluorescence in-situ hybridization (FISH) after bacterial lysis. In some embodiments, measuring microbial viability in droplets is performed using a technology that does not affect viability of the microorganism, including measurement of solution turbidity, pH or fluorescence of a metabolically active dye. In some embodiments, measuring microbial viability includes obtaining a measure of microbial viability from a discrete subset of microdroplets from a first population of microdroplets from a first portion of the sample measured at a first time point, and obtaining a measure of microbial viability from a discrete subset of microdroplets from a second population of microdroplets from the first portion of the sample measured at a second time point. In some embodiments, the measure of microbial viability is whether an indicator of microbial viability exceeds a preset threshold. In some embodiments, the individual subset of microdroplets includes one or more microdroplets. In some embodiments, the one or more portions of samples are cultured in a culture medium. In some embodiments, the culture medium is added before formation of microdroplets or during formation of microdroplets. In some embodiments, the method further includes immobilizing the one or more populations of microdroplets encapsulating microbes on an indexed array. In some embodiments, the method further includes flowing the one or more populations of microdroplets encapsulating microbes through a high throughput microdroplet reader. In some embodiments, the different concentration of antimicrobial spans a desired clinical range in the range of 0.002 mg/L to 500 mg/L. In some embodiments, measuring microbial viability includes measuring a fluorescence signal of a label. In some embodiments, the fluorescence is measured using a fluorescence reader. In some embodiments, microbial viability is determined by measuring absorbance or electrochemical properties of a viability indicator dye. In some embodiments, the viability indicator dye includes resazurin, formazan, or analogues or salts thereof. In some embodiments, microbial viability is determined by measuring absorbance or electrochemical properties of a viability indicator, or by measuring pH or turbidity. In some embodiments, an average number of microbes per microdroplet is less than 2. In some embodiments, an average number of microbes per microdroplet is less than 1. In some embodiments, the microbe is a bacteria. In some embodiments, the bacteria is E. coli, P. aeruginosa, S. aureus, S. epidermidis, E. faecalis, K. pneumoniae, E. cloacae, A. baumanii, S. marcescens, or E. faecium. In some embodiments, the microbes are bacteria and wherein the antimicrobial is an antibiotic. In some embodiments, the antibiotic is an aminocoumarin, an aminoglycoside, an ansamycin, a carbacephem, a carbapenem, a cephalosporin, a glycopeptide, a lincosamide, a lipopeptide, a macrolide, a monobactam, a nitrofuran, a penicillin, a polypeptide, a quinolone, a streptogramin, a sulfonamide, or a tetracycline, or a combination thereof. In some embodiments, the antibiotic is ampicillin. In some embodiments, the microdroplet includes an oil phase and a surfactant phase. In some embodiments, the microdroplets are formulated by microfluidic channels, agitation, electric forces, or membrane filtration. In some embodiments, the one or more populations of microdroplets are formulated as stable water-in-oil emulsions. In some embodiments, determining susceptibility of the microbes to the antimicrobial is completed more quickly than when no microdroplets are formed. In some embodiments, determining susceptibility of the microbes to the antimicrobial is completed in a time within a range of 3-24 hours, 3-20 hours, 3-15 hours, 3-8 hours, 5-20 hours, 5-15 hours, or 5-8 hours. In some embodiments, determining susceptibility of the microbes to the antimicrobial is completed in not more than 24 hours; in not more than 15 hours; in not more than 12 hours; in not more than 10 hours; in not more than 8 hours; in not more than 5 hours; in not more than 3 hours. In some embodiments, the sample is whole blood, positive blood culture, peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen (including prostatic fluid), Cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates or other lavage fluids, blastocoel cavity, umbilical cord blood, or maternal circulation.

### BRIEF DESCRIPTION OF THE DRAWINGS

FIG. 1 shows a schematic representation of an embodiment of a method of assessing microbial viability on a discrete subset of microdroplets.

FIG. 2 shows a graphical representation of an embodiment of a measurement of microbial viability of the method of FIG. 1, showing microdroplet fluorescence intensity as a function of time including microdroplets without antibiotic or with antibiotics at a concentration less than a minimum inhibitory concentration (MIC; solid line) compared to microdroplets with antibiotic at a concentration of greater than a MIC (dashed line).

FIGS. 3A-3D depict results of an embodiment of microdroplet antibiotic susceptibility testing using a fluorescent viability indicator. FIG. 3A depicts change in fluorescent intensity in microdroplets at 1-hour, 2-hour, and 3-hour time points in samples without antibiotic (condition A) and samples with antibiotic at two times MIC (condition B). FIG. 3B depicts micrographs of microdroplets under the conditions of FIG. 3A. FIG. 3C shows a micrograph of microdroplets containing E. coli without antibiotics. FIG. 3D shows a micrograph of microdroplets containing E. coli and antibiotic at two times MIC.

FIG. 4 shows a schematic representation of an embodiment of a method of assessing microbial viability on a plurality of microdroplets.

FIG. 5 shows a schematic representation of an embodiment of a method of assessing microbial viability on microdroplets based on measuring microbial viability in microdroplets that exceed a predetermined threshold.

FIG. 6 shows a graphical representation of an embodiment of a measurement of microbial viability of the method of FIG. 5, showing microbial viability in microdroplets that exceed a threshold as a function of time, including microdroplets without antibiotic or with antibiotics at a concentration less than MIC (solid line) compared to microdroplets with antibiotic at a concentration of greater than a MIC (dashed line).

FIG. 7 shows a schematic representation of an embodiment of a method of assessing microbial viability by incubating a sample in antibiotic and preparing microdroplets at each measurement point.

FIG. 8 shows a graphical representation of an embodiment of a measurement of microbial viability of the method outlined in FIG. 7, showing microbial viability in microdroplets that exceed a threshold as a function of time including microdroplets without antibiotic or with antibiotics at a concentration less than MIC (solid line) compared to microdroplets with antibiotic at a concentration of greater than a MIC (dashed line).

### DETAILED DESCRIPTION

Increasing antimicrobial resistance and a dwindling antimicrobial pipeline have created a global public health crisis in which an increasing number of patients are infected with antimicrobial-resistant bacteria. Appropriate antimicrobial therapy that can be instated within hours of the onset of infection can positively impact patient outcome. However, the current practice for identifying an infection requires excessive time. Accordingly, there is a strong need for a more rapid antimicrobial susceptibility testing, preferably one that can identify specific antimicrobial susceptibilities within hours after blood samples are drawn. A rapid test of this type would therefore permit physicians to initiate the optimal drug therapy from the start, rather than starting with a suboptimal or completely ineffective antimicrobial, hence greatly increasing clinical responsiveness. Major efforts at improving the current antimicrobial susceptibility testing (AST) practices are aimed at reducing the target time to result (TTR).

In microdroplet-based microbial identification (ID) and antimicrobial susceptibility testing (AST), clinical specimens are partitioned into small volume microdroplets, each of which contains a small number of organisms, typically 1 to 5 organisms per microdroplet. This approach allows high effective concentrations of organisms within each occupied microdroplet, and may enable rapid time-to-result as well as direct-from-specimen testing of specimens with polymicrobial infections because each microbe is segregated into independent microdroplets.

A challenge for microdroplet-based ID/AST technologies is that a large number of microdroplets are generated, and many of these microdroplets may not contain any organismal cells. Thus, large numbers of microdroplets must be evaluated in order to obtain clinically relevant results.

Accordingly, some embodiments provided herein relate to methods of determining susceptibility of a microbe to an antimicrobial by providing a sample having microbes, encapsulating microbes from the sample within microdroplets, where the sample is divided into portions before or after forming the microdroplets, contacting each portion of the sample with a different concentration of antimicrobial either before or after formation of microdroplets, and measuring microbial viability of the microbe within the microdroplet. This method described herein may be altered, modified, or varied according to the embodiments, methods, systems and modes described herein in further detail.

Some of the embodiments, methods, and modes described herein include one or more advantages, including, for example: direct-from-specimen methods that have no need for specimen culturing to obtain an isolated colony; increased bacteria concentration due to the partitioning of samples in microdroplets resulting in high starting concentration in occupied microdroplets; and rapid growth detection where discrete proliferation events can be detected.

Without wishing to be bound by a theory, embodiments of the antimicrobial susceptibility testing method described herein can rapidly detect microbial infection of a sample caused by different pathogens (e.g., bacteremia, fungemia, viremia) and provide the antimicrobial sensitivity and resistance profile of the causative agent (e.g., microbial pathogen).

Furthermore, advantages of embodiments of microdroplet based AST disclosed herein over existing AST methods includes the potential to handle direct-from-specimen (low titer) and/or poly-microbial (mixed infection) samples. Both of these advantages arise primarily due to the partitioning of clinical specimen into very small volume microdroplets, each of which can be tailored to contain no more than a single organism and each of which can be addressed individually. Embodiments of AST using microdroplets as described herein may be performed directly from clinical specimen (without the need for culturing on a solid medium) using microdroplets, resulting in significant savings in time to result (TTR). Embodiments partitioning bacteria into small volumes also allow high effective concentrations of organisms, which results in faster reaction kinetics thereby further improving TTR.

An additional advantage of embodiments performing AST using microdroplets is the improved TTR for testing “delayed resistance” bacterial phenotypes. These bacteria drug combinations are particularly challenging for both current and emerging AST technologies due to the lengthy TTR required to correctly identify the bacteria as resistant.

Accordingly, some embodiments provided herein relate to novel compositions, methods, systems, and/or kits for determining a minimum inhibitory concentration (MIC) of an antimicrobial. In some embodiments, a method for determining antimicrobial MIC is performed by exposing a sample having microbes to a concentration of an antimicrobial, encapsulating microbes within microdroplets, and measuring microbial viability of microbes within the microdroplets.

As described herein, variations of the method may be performed based on the preparation of microdroplets, the measurement of microbial viability in the microdroplets, and/or the steps for measuring microbial viability. Some variations of the methods are described herein as modes. It will be understood by one of skill in the art that additional variations and/or modes may be performed, and that in some embodiments, aspects of any given mode may be interchanged, replaced, or substituted, with aspects from a different mode. Furthermore, in some embodiments, various aspects of any given mode may be removed, added, revised, or otherwise varied.

### Mode 1—Measuring Microbial Viability in a Population of Microdroplets

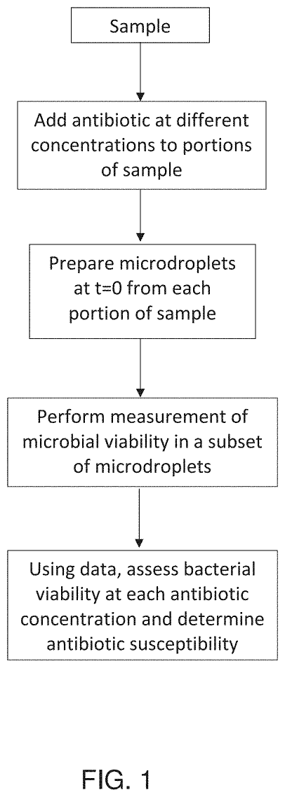

Some embodiments provided herein relate to a first mode for determining microbial viability. A first mode for determining microbial viability is schematically depicted in FIG. 1. As described herein, a first mode for determining microbial viability includes providing a sample having microbes therein, dividing the sample into a number of portions, preparing microdroplets encapsulating microbes from the sample, either before or after the portions are formed (FIG. 1 depicts forming portions first), contacting each portion to a different concentration of an antimicrobial of interest either before or after preparing the microdroplets (FIG. 1 depicts adding an antibiotic prior to forming microdroplets), and measuring viability of the microbes by measuring a signal of microbial viability. Measuring a signal of microbial viability may be performed by obtaining a measure of microbial viability from a discrete subset of microdroplets, or a plurality of discrete subsets of microdroplets, from a population of microdroplets. The results for the measurements from discrete subsets of microdroplets at a first time point can then be combined to generate a result for the portion of the sample from which the microdroplet(s) were taken at this first time point. This process can be repeated at additional time points to determine microbial viability in that portion over time, thereby determining the susceptibility of the microbe to a given concentration of antimicrobial. Typically, it is not necessary to measure the exact same population of microdroplets at the first and subsequent time points, as long as the population of microdroplets measured at each time point are representative of the portion of the sample from which they are taken. The results from the various portions exposed to different concentrations of antimicrobial can then be used to determine the susceptibility of the microbe to the antimicrobial. A determination of antimicrobial susceptibility can be made upon determining that the measure of microbial viability does not increase over time, indicating that no growth of microbes occurs over time when contacted with the concentration of antimicrobial present in the portion of the sample from which the measurements were made. Conversely, an increase in the measure of microbial viability over time indicates growth of microbes in the concentration of antimicrobial present in the portion of the sample from which the measurements were made. A minimum inhibitory concentration can be determined utilizing the results of antimicrobial susceptibility and/or microbe growth from the several portions of the sample exposed to different concentrations of antimicrobial.

In some embodiments, preparing microdroplets from each portion results in a population of microdroplets in each portion. In some embodiments, the size of the microdroplets are a size sufficient to encapsulate a microbe of interest, for example a size within a range from about 2 μm to about 500 μm, 2 μm to 200 μm, 2 μm to 50 μm, 2 μm to 10 μm, 10 μm to 200 μm, 10 μm to 50 μm, 50 μm to 200 μm, or 50 μm to 100 μm. The size of microdroplets and methods for preparing the same are described in additional detail herein. For example, in some embodiments, a first portion of the sample is contacted with a first concentration of antimicrobial and one or more populations of microdroplets are formed therefrom; a second portion of the sample is contacted with a second concentration of antimicrobial and one or more populations of microdroplets are formed therefrom; and so forth for a desired number of portions, each portion having a different concentration of antimicrobial to be tested. Alternatively, the microdroplets could be formed from the portions prior to contacting the portion with the antimicrobial. The concentrations of antimicrobial are typically selected to include a range of concentrations of antimicrobial that includes a concentration of antimicrobial that is or is suspected of being the minimum inhibitory concentration (MIC), as described in more detail herein. In some embodiments, one or more of the portions of sample is not exposed to any antimicrobial (antimicrobial concentration of zero), e.g. for purposes of a control.

In some embodiments, the first mode includes measuring microbial viability including obtaining a measure of microbial viability from a discrete subset of microdroplets from a first population of microdroplets from a portion of the sample measured at a first time point and obtaining a measure of microbial viability from a discrete subset of microdroplets from a second population of microdroplets from the same portion of the sample at a second time point to determine a change in a signal of microbial viability over time. In some embodiments, measurements are obtained from a plurality of discrete subsets of microdroplets at the first and/or second time points. A subset of microdroplets includes one, or more than one microdroplet, for example, a subset includes, or includes at least, 1, 2, 3, 4, 5, 6, 7, 8, 9, 10, 20, 30, 40, 50, 60, 70, 80, 90, 100, 200, 300, 400, 500, 600, 700, 800, 900, 1000, 1500, 2000, 3000, 4000, 5000, 6000, 7000, 8000, 9000, or 10000 microdroplets, or a range defined by any two of the preceding values, for example 1-5, 1-10, 5-10, 5-20, 10-50, 10-100, 50-100, 50-500, 100-500, 100-1000, 500-1000, 500-5000, 1000-5000, 1000-10000, or 5000-10000 microdroplets. In some embodiments, a measure of microbial viability may be performed by measuring additional discrete subsets of microdroplets at additional time points, such as, for example, a third time point, a fourth time point, a fifth time point, and so forth for a given number of time points sufficient to determine microbial viability microdroplet. For example, in some embodiments, a measure of microbial viability is obtained at a third time point from a third population of microdroplets obtained from the same portion of the sample. Additional time points and measurement of microbial viability for populations of microdroplets may be performed as required for any given assay, such as for example, a fourth time point from a fourth population of microdroplets, a fifth time point from a fifth population of microdroplets, a sixth time point from a sixth population of microdroplets, a seventh time point from a seventh population of microdroplets, an eighth time point from an eight population of microdroplets, a ninth time point from a ninth population of microdroplets, a tenth time point from a tenth population of microdroplets, or more. In some embodiments, the time points at which the measurement of microbial viability is selected in the range from time 0 (e.g., the time at which the microdroplets encapsulating microbes are formed) to time 24 hours. Thus, in some embodiments, the time points include a measurement at 0, 0.5, 1, 2, 3, 4, 5, 6, 7, 8, 9, 10, 11, 12, 13, 14 or 15 minutes or 0.5, 0.75, 1, 1.5, 2, 2.5, 3, 3.5, 4, 4.5, 5, 5.5, 6, 7, 8, 9, 10, 12, 15, 18, 21, or 24 hours, or an amount of time within a range defined by any two of the aforementioned values. In some embodiments, the time points include measurement at any given frequency from 0 to 15 minutes, 0 to 10 minutes, 0 to 5 minutes, 0 to 1 minutes, 5 to 15 minutes, 5 to 10 minutes, or 10 to 15 minutes, 0 to 24 hours, 0 to 21, 0 to 18, 0 to 15, 0 to 12, 0 to 10, 0 to 6, 0 to 5 hours, 0 to 4 hours, 0 to 3 hours, 0 to 2 hours, 0 to 1 hours, 0 to 0.5 hours, 0.25 to 6 hours, 0.25 to 5 hours, 0.25 to 4 hours, 0.25 to 3 hours, 0.25 to 2 hours, 0.25 to 1 hour, 0.25 to 0.5 hours, 1 to 6 hours, 1 to 5 hours, 1 to 3 hours, or 1 to 2 hours, or until a determination of antimicrobial susceptibility can be realized. In some embodiments, antimicrobial susceptibility is made from a time of contacting a sample with an antimicrobial to making a determination of antimicrobial susceptibility in no more than 24, 20, 15, 10, 8, 5, 3, or 1 hours, or an amount of time sufficient to make a determination that the microbe is susceptible and/or not susceptible to the tested antimicrobial. In some embodiments, antimicrobial susceptibility is made from a time of contacting a sample with an antimicrobial to making a determination of antimicrobial susceptibility in not more than 24 hours; in not more than 15 hours; in some it is not more than 12 hours; in some it is not more than 10 hours; in some it is not more than 8 hours; in some it is not more than 5 hours; in some it is not more than 3 hours; in some it is not more than 1 hour. In some embodiments, the determination of whether the microbe is susceptible and/or not susceptible to the antimicrobial is completed more quickly using the microdroplet susceptibility testing described herein than when testing antimicrobial susceptibility when not formed in microdroplets. This process can be repeated for additional portions of the sample. In some embodiments this process is repeated for a second, a third, a fourth, a fifth, or more portions of the sample.

Once the measures of viability are obtained at the first, second, and subsequent time points for a given portion of the sample (e.g., a first, second, third, etc. portion), these measures can be compared to determine viability over time for each portion. In some embodiments, the discrete measurements at each of the various time points are aggregated so that aggregate measurement at each time point can be compared to each other. For example, in one embodiment an average of the measure of microbial viability from a plurality of discrete subsets of microdroplets measured at the first time point is compared to an average of the measure of microbial viability from a plurality of discrete subsets of microdroplets measured at the second time point. In some embodiments, rather than aggregating the data from measurements of several discrete subsets before comparison over time points, discrete data points from subsets are compared over time, and the comparisons from the discrete data points over time are aggregated to obtain a comparison. For example, in one embodiment the measure of microbial viability from a discrete subset of microdroplets measured at the first time point is compared to the measure of microbial viability from a discrete subset of microdroplets measured at the second time point for a plurality of subsets of microdroplets measured at the first and second time points, and optionally the plurality of discrete comparisons are averaged to obtain a comparison between the first and second time point.

In some embodiments, the individual microdroplets in the subset(s) and/or first population measured at the first time point are not the same microdroplets in the subset(s) and/or second population measured during the second or subsequent time points. While there can be overlap between the individual microdroplets in the subset(s) and/or first population measured at the first time point, and the individual microdroplets in the subset(s) and/or population measured at the second or subsequent time points, it is not necessary that exactly the same individual microdroplets are measured at each time point. Thus, in some embodiments one or more discrete subset(s) of microdroplets from the first population are not in the second population, and one or more discrete subset(s) of microdroplets from the second population are not in the first population, and so forth for third, fourth, fifth and any subsequent populations and time points. In other embodiments, the same discrete subset(s) of microdroplets is measured at the first and second time point, and any subsequent time points, such that the discrete subset(s) of microdroplets from the first population are the same discrete subset(s) of microdroplets from the second and subsequent populations. In some embodiments the measurements of microbial viability obtained at the first, second, and any subsequent time points are not assigned to discrete subsets of microdroplets, whether or not the same discrete subset(s) of microdroplets are measured at each time point.

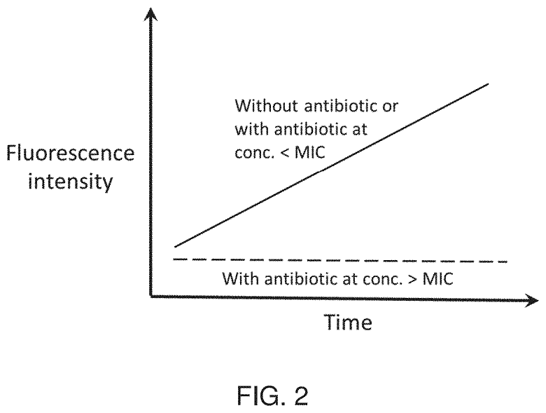

As shown in the embodiment in FIG. 2, a measure of microbial viability may be made over time by determining fluorescence intensity, for example, fluorescence intensity of resazurin. A measurement of fluorescence intensity is shown for microdroplets exposed to an antimicrobial below a minimum inhibitory concentration (MIC) or with no antimicrobial added (solid line) and for microdroplets exposed to an antimicrobial above a MIC (dashed line). The solid line indicates increased fluorescence intensity due to increased microbial growth over time, due to an absence of antimicrobial or presence of antimicrobial at a concentration less than the MIC. The dashed line indicates no or insignificant increase in fluorescence intensity due to no microbial growth over time as a result of antimicrobial concentration greater than MIC.

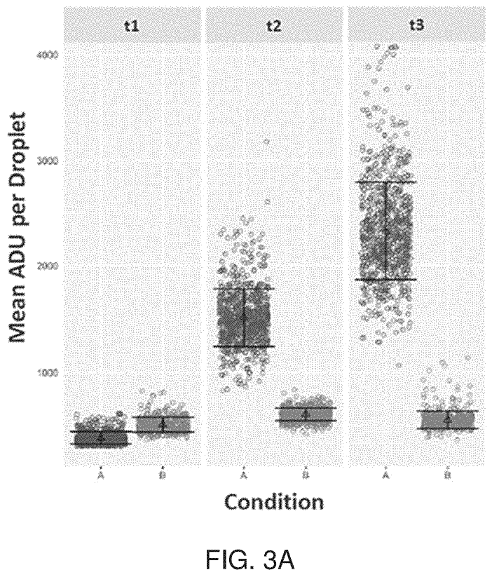

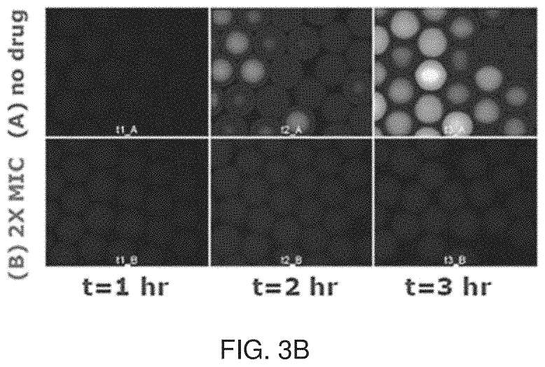

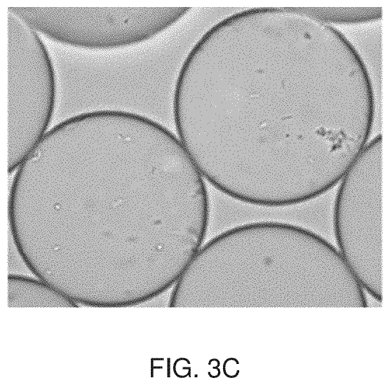

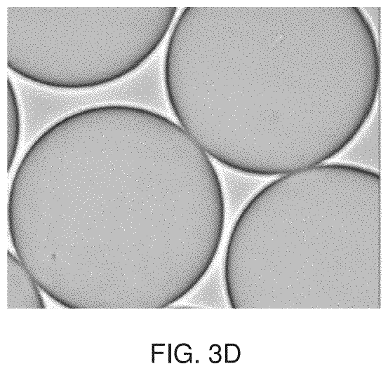

FIG. 3 illustrates results of measurement of microbial viability using an embodiment of the method set forth in Mode 1. FIG. 3A depicts microdroplet AST using a fluorescent viability indicator for E. coli encapsulated within microdroplets. The microdroplets encapsulating E. coli were prepared from a first portion of the sample that was not exposed to antibiotic (Condition A), or from a second portion of the sample that was exposed to antibiotic at a concentration of 2×MIC (Condition B). The antibiotic used in this example was ampicillin. The microdroplets were formed and incubated for the specified time, including 1 hour (t1), 2 hours (t2), and 3 hours (t3). FIG. 3A depicts plots of measurements from discrete subsets of microdroplets (single microdroplets in this case) from three populations of microdroplets at the three time points (t1, t2, t3), taken from the two portions of the sample (Condition A or B). The triangles represent the mean for each subset, with the error bars indicating one standard deviation of the mean. FIG. 3B depicts micrographs of representative microdroplets from each population at each time point, from each of Condition A and B. At time 1 hour, the fluorescence intensity remained low for the microdroplets in both Condition A and B. At time 2 hours and 3 hours, the fluorescence intensity increased for the microdroplets in Condition A, indicative of E. coli growth due to the absence of antibiotic, whereas the fluorescence intensity remained low for microdroplets in Condition B, indicative of no E. coli growth due to the presence of 2×MIC ampicillin. FIG. 3C shows micrographs of microdroplets that are not exposed to antibiotic, and show the presence of E. coli when no antibiotic is present (Condition A). FIG. 3D shows micrographs of microdroplets exposed to 2×MIC ampicillin (Condition B), and no E. coli is present.

### Mode 2—Measuring Microbial Viability in Discrete Subsets of Microdroplets Over Time

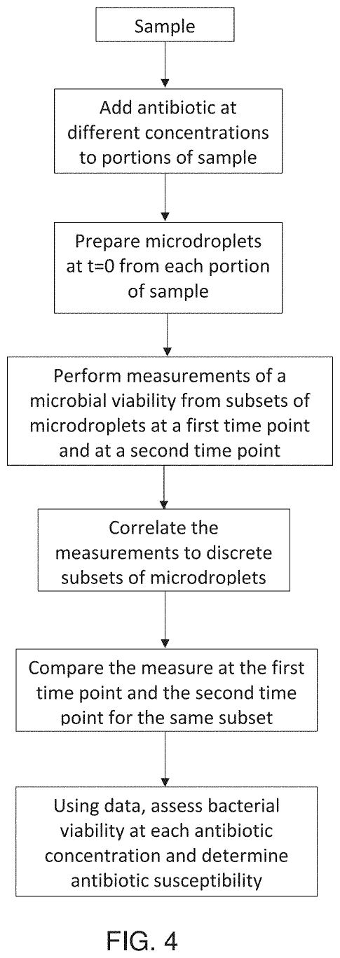

Some embodiments provided herein relate to a second mode for determining microbial viability, which is related to Mode 1, as well as the other modes disclosed herein. Thus, the disclosure regarding the other modes, including but not limited to Mode 1, is applicable to Mode 2 as well. An embodiment of Mode 2 for determining microbial viability is schematically depicted in FIG. 4. As described for Mode 1, the second mode for determining microbial viability includes providing a sample having microbes therein, dividing the sample into a number of portions, preparing microdroplets encapsulating microbes from the sample, either before or after the portions are formed (FIG. 4 depicts forming portions first), contacting each portion to a different concentration of an antimicrobial of interest either before or after preparing the microdroplets (FIG. 4 depicts adding the antimicrobial prior to forming microdroplets), and measuring viability of the microbes by measuring a signal of microbial viability in discrete subsets of microdroplets from populations of microdroplets at first, second, and optionally additional time points. As disclosed herein, time points may be selected at various frequencies and over ranges of time. Furthermore, as disclosed herein, time points are selected to allow sufficient time for a determination that the microbe is susceptible and/or not susceptible to an antimicrobial. In some embodiments, a determination of whether the microbe is susceptible and/or not susceptible to the antimicrobial is completed more quickly using the microdroplet susceptibility testing described herein than when testing antimicrobial susceptibility when not formed in microdroplets, (e.g., in not more than 24 hours; in not more than 15 hours; in not more than 12 hours; in not more than 10 hours; in not more than 8 hours; in not more than 5 hours; in not more than 3 hours; in not more than 1 hour). Embodiments of Mode 2 include assigning the signal of microbial viability from the population of microdroplets to the discrete subset(s) of microdroplets, so that the results from a particular discrete subset of microdroplets (e.g. single microdroplets) at a first time point can be compared to the results from that same discrete subset of microdroplets (e.g. single microdroplets) at a second time point across the population of microdroplets. Typically, for each portion of the sample exposed to a different concentration of antimicrobial, measurements are taken from a sufficient number of discrete subsets of microdroplets to ensure that a representative number of microdroplets are measured for each portion of the sample. The results for each discrete subset of microdroplets over time can then be combined to generate a result for the portion of the sample from which the microdroplets were taken, and the results from the various portions exposed to different concentrations of antimicrobial are used to determine the susceptibility of the microbe to the antimicrobial.

In an embodiment of Mode 2, a sample comprising microbes is separated into one or more portions of samples comprising microbes, and the one or more portions of sample are contacted with an antimicrobial, each of the one or more portions of samples being contacted with a different concentration of antimicrobial. One or more populations of microdroplets encapsulating microbes are then formed from the one or more portions of samples. The viability of microbes encapsulated within microdroplets is then measured. An embodiment of Mode 2 comprises obtaining a measure of microbial viability from discrete subsets of microdroplets in a first population of microdroplets from a first portion of the sample measured at a first time point, and obtaining a measure of microbial viability from discrete subsets of microdroplets in a second population of microdroplets from the first portion of the sample measured at a second time point, further comprising assigning measurements obtained at the first and second time points to discrete subsets of microdroplets, wherein at least some of the discrete subsets of microdroplets in the first population are the same discrete subsets of microdroplets in the second population such that the measurement of microbial viability obtained for a discrete subset of microdroplets at the first time point can be compared to the measurement of microbial viability obtained for that same discrete subset of microdroplets obtained at the second time point. Some embodiments involve, for at least a plurality of subsets of microdroplets, actually making a comparison of the measurement of microbial viability obtained for a discrete subset of microdroplets at the first time point to the measurement of microbial viability obtained for that same discrete subset of microdroplets obtained at the second time point (and any additional time points). As stated herein, a subset of microdroplets includes one, or more than one microdroplet, for example, a subset includes, or includes at least, 1, 2, 3, 4, 5, 6, 7, 8, 9, 10, 20, 30, 40, 50, 60, 70, 80, 90, 100, 200, 300, 400, 500, 600, 700, 800, 900, 1000, 1500, 2000, 3000, 4000, 5000, 6000, 7000, 8000, 9000, or 10000 microdroplets, or a range defined by any two of the preceding values, for example 1-5, 1-10, 5-10, 5-20, 10-50, 10-100, 50-100, 50-500, 100-500, 100-1000, 500-1000, 500-5000, 1000-5000, 1000-10000, or 5000-10000 microdroplets.

As discussed herein for Mode 1, additional populations of microdroplets and subsets thereof can be measured at additional time points (e.g., third, fourth, fifth, sixth, seventh, etc.). For some embodiments of Mode 2, the measurements obtained at the first, second and any subsequent time points are assigned to discrete subsets of microdroplets, and at least one or more of the discrete subsets of microdroplets in the first population are the same discrete subsets of microdroplets in the second and any subsequent populations such that the measurement of microbial viability obtained for a discrete subset of microdroplets (e.g., single microdroplets) at the first time point can be compared to the measurement of microbial viability obtained for that same discrete subset of microdroplets obtained at the second and any subsequent time point(s) (e.g., third, fourth, fifth, sixth, etc.). In some embodiments, because not every microdroplet in the first and second, or any subsequent populations, must be utilized to make a comparison, at least one discrete subset of microdroplets in the first population is not in the second population, or any subsequent population, and at least one discrete subset of microdroplets in the second population, or any subsequent population, is not in the first population.

In some embodiments including but limited to those of Mode 2, measuring a discrete subset of microdroplets at a first time point and at subsequent time points may include indexing the discrete subsets of microdroplets. The indexing can be used, for example, to assign the measure of microbial viability to a particular subset of microdroplets, or to ensure a representative number of subsets of microdroplets are measured for a portion of the sample. In some embodiments, the microdroplets are deposited on an indexed array, and measurements of microbial viability are performed on the indexed array. Indexing may be performed by methods known in the art. For example, indexing may be performed in some embodiments by incorporating optical and/or magnetic reporters (including, for example, organic fluorophores, quantum dots, SERS tags) within each sample microdroplet.

Mode 3—Measuring Microbial Viability in Microdroplets when Signal Exceeds Threshold

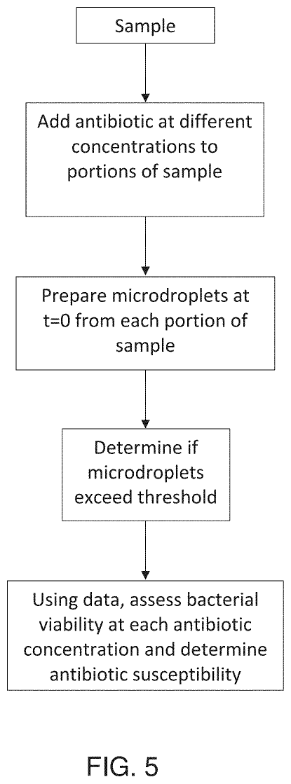

Some embodiments provided herein relate to a third mode for determining microbial viability, which is related to the other modes disclosed herein, e.g., Mode 1 and 2. Thus, the disclosure with respect to other modes, including but not limited to Modes 1 and 2 are applicable to Mode 3 as well. An embodiment of a third mode for determining microbial viability is depicted in FIG. 5. As described for Mode 1, a third mode for determining microbial viability includes providing a sample having microbes therein, dividing the sample into a number of portions, preparing microdroplets encapsulating microbes from the sample, either before or after the portions are formed (FIG. 5 depicts forming portions first), contacting each portion to a different concentration of an antimicrobial of interest either before or after preparing the microdroplets (FIG. 5 depicts adding an antibiotic prior to forming microdroplets), and measuring viability of the microbes by measuring a signal of microbial viability. Measuring a signal of microbial viability may be performed by measuring microbial viability in one or more discrete subset(s) of microdroplets (e.g. a single microdroplet) from a population of microdroplets. Embodiments of Mode 3 include measuring whether the signal of microbial viability exceeds a threshold value in discrete subset(s) of microdroplets (e.g. single microdroplets), as opposed to only measuring the level or amount of signal. Thus, in this sense the measure of microbial viability is digital—either exceeding or not exceeding the threshold. This permits measuring the change in the amount or number of subset(s) of microdroplets in a portion of the sample that are exceeding the threshold over time. If that amount or number remains constant or does not increase with time, the microbes in that portion are not viable and thus susceptible to the antimicrobial concentration in that portion of the sample. In contrast, if the number or amount of subset(s) of microdroplets exceeding the threshold increases over time before reaching a saturation level, it indicates that the microbes are viable at the concentration of antimicrobial in that portion of the sample.

In some embodiments, measuring microbial viability includes obtaining a measure of microbial viability from a discrete subset of microdroplets in a first population of microdroplets from a first portion of the sample measured at a first time point, and obtaining a measure of microbial viability from a discrete subset of microdroplets in a second population of microdroplets from the first portion of the sample measured at a second time point, wherein the measure of microbial viability is whether an indicator of microbial viability exceeds a preset threshold. In some embodiments, a preset threshold is exceeded when a measure of microbial viability exceeds an amount determined to indicate the microbe is viable. For example, a threshold may include value wherein when exceeded, the growth of microbes has exceeded an amount that would indicate that the antimicrobial (and antimicrobial concentration) being tested is less than a minimum inhibitory concentration (MIC). In some embodiments, a threshold is exceeded when an indicator reaches a determined measure of fluorescence. A subset of microdroplets includes one, or more than one microdroplet, for example, a subset includes, or includes at least, 1, 2, 3, 4, 5, 6, 7, 8, 9, 10, 20, 30, 40, 50, 60, 70, 80, 90, 100, 200, 300, 400, 500, 600, 700, 800, 900, 1000, 1500, 2000, 3000, 4000, 5000, 6000, 7000, 8000, 9000, or 10000 microdroplets, or a range defined by any two of the preceding values, for example 1-5, 1-10, 5-10, 5-20, 10-50, 10-100, 50-100, 50-500, 100-500, 100-1000, 500-1000, 500-5000, 1000-5000, 1000-10000, or 5000-10000 microdroplets.

The comparison of the plurality of measurements obtained at the first and second time points (and any subsequent time points) can take several forms. For example, the same subset of microdroplets could be monitored over time to determine if the subset goes from not exceeding to exceeding the threshold. This could be done for a plurality of subsets. Alternatively, the number or percentage of subsets in a plurality of subsets exceeding the threshold could be monitored over time to determine if the number or percentage is increasing. In this case, the same exact subsets need not be monitored at the first, second, and any subsequent time points, although the subsets being monitored could be partially or exactly the same subsets. As disclosed herein, time points may be selected at various frequencies and over ranges of time. Furthermore, as disclosed herein, time points are selected to allow sufficient time for a determination that the microbe is susceptible and/or not susceptible to an antimicrobial, for example, when the signal of microbial viability exceeds a threshold. In some embodiments, a determination of whether the microbe is susceptible and/or not susceptible to the antimicrobial is completed more quickly using the microdroplet susceptibility testing described herein than when testing antimicrobial susceptibility when not formed in microdroplets (e.g., in not more than 24 hours; in not more than 15 hours; in not more than 12 hours; in not more than 10 hours; in not more than 8 hours; in not more than 5 hours; in not more than 3 hours; in not more than 1 hour). In some embodiments, a composite of the measure of microbial viability from a plurality of discrete subsets of microdroplets measured at a first time point is compared to a composite of the measure of microbial viability from a plurality of discrete subsets of microdroplets measured at a second and/or subsequent time points. In some embodiments, the composite of the measure of microbial viability is the percentage of the plurality of discrete subsets of microdroplets measured at a time point that exceeds the threshold. Some embodiments comprise comparing the measure of microbial viability from a discrete subset of microdroplets obtained at a first time point to the measure of microbial viability from a discrete subset of microdroplets obtained at a second time point, and any subsequent time points, for a plurality of subsets of microdroplets measured at the first, second and any subsequent time points. In some embodiments, the measurements of microbial viability obtained at the first, second and any subsequent time points are not assigned to discrete subsets of microdroplets. In some embodiments, they are assigned, and some embodiments comprise comparing the measure of microbial viability obtained for a discrete subset of microdroplets at the first time point to the measure of microbial viability obtained for that same discrete subset of microdroplets obtained at the second, and any subsequent time point, for a plurality of subsets of microdroplets. In some embodiments, one or more discrete subsets of microdroplets from the first population are not in the second population, and one or more discrete subsets of microdroplets from the second population are not in the first population, and so forth for any additional populations.

As used herein, the term “assign” or “assignment” refers to attributing a measure of microbial viability to a microdroplet or to a discrete population of microdroplets, wherein the measure of microbial viability may correspond to a specific microdroplet or to a specific discrete population of microdroplets.

A measurement of microbial viability may be performed as described herein, including but not limited to as described for Mode 1 or Mode 2, including measurements of microbial viability for a population of microdroplets and/or for a discrete subset of microdroplets. The measurement of microbial viability may be performed continuously or periodically over time, until a signal of microbial viability exceeds a preset threshold. In some embodiments, for each discrete subset of microdroplets or population of microdroplets, a number or percentage of subsets of microdroplets that exhibit a measure of microbial viability, such as a measure of fluorescence intensity, above a predetermined threshold is determined. For example, the number of subsets of microdroplets exceeding the predetermined threshold can be determined by scanning subsets of microdroplets (e.g. single, 2, 3, 4 or more microdroplets per subset) with a high throughput microdroplet analyzer and counting distinct detector events where microbial viability measures, such as fluorescence intensity, does and/or does not exceed a preset threshold. A number of subsets of microdroplets exceeding the threshold value compared to the total number of subsets of microdroplets evaluated provides a percentage of microbial viability for a given population of microdroplets (e.g., for a given concentration of antimicrobial). In some embodiments, determining a percentage of microbial viability for a population of microdroplets is repeated at various time points to determine microbial viability for the population of microdroplets as a function of time for each portion of the sample having the same concentration of antimicrobial. The microbial viability is then assessed and the MIC is determined from the measurements across antimicrobial concentrations.

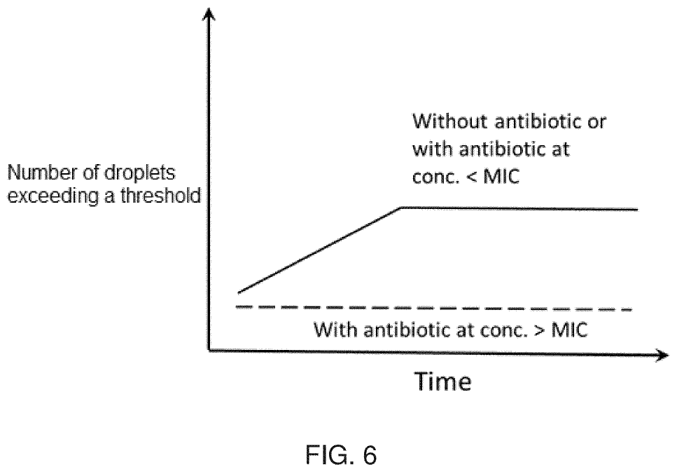

An embodiment of measuring microbial viability by the method of Mode 3 is depicted in FIG. 6, by determining microbial viability based on whether a measure exceeds a threshold. As shown in FIG. 6, the microbial viability is measured as a function of time by determining a measure of microbial viability and whether the measure of microbial viability exceeds a predetermined threshold. Microdroplets that are not exposed to antimicrobial or are exposed to antimicrobial below the MIC increase in fluorescence intensity over time. Once the signal of fluorescence intensity exceeds a preset threshold, an indication of microbial viability is determined (solid line). In contrast, any signal that does not exceed the threshold is indicative of no viability. For example, microdroplets exposed to antimicrobial above a MIC does not exceed a preset threshold (dashed line), and a determination of no microbial viability is assessed.

### Mode 4—Forming Microdroplets at Measurement Time Points

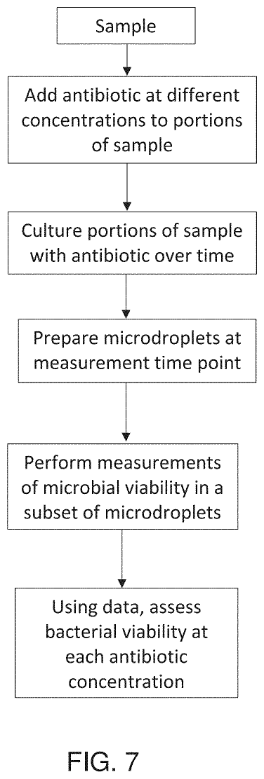

Some embodiments provided herein relate to a fourth mode for determining microbial viability, which is related to the other modes disclosed herein, e.g., Modes 1, 2, and 3. Thus, the disclosures with respect to the other modes, including but not limited to Modes 1, 2, and 3 are applicable to Mode 4 as well. An embodiment of a fourth mode for determining microbial viability is depicted in FIG. 7. As described for Mode 1, a fourth mode for determining microbial viability includes providing a sample having microbes therein, dividing the sample into a number of portions, exposing each portion to a different concentration of an antimicrobial of interest. In embodiments of Mode 4, microdroplets are not immediately prepared. Instead, the microbes in each portion of the sample having a different concentration of antimicrobial are cultured for a period of time and the microdroplets are prepared from each portion at various measurement time points. A measure of microbial viability of microbes is then performed by measuring a signal of microbial viability of one or more discrete subset(s) of microdroplets from a population of microdroplets, as described herein (including but not limited to embodiments of Modes 1-3). Thus, in Mode 4, the portion of sample is cultured over a period of time prior to forming microdroplets, which are formed at various measurement time points. There can be several reasons for culturing the portions of the sample and making the microdroplets at the various measurement time points. For example, some microbes lose or acquire resistance to an antimicrobial depending on the density of the microbe in the culture (e.g. due to quorum sensing). These effects may not be observed if the microbe is cultured in a microdroplet environment. Another reason is that by maintaining the culture and making microdroplets from the culture as needed at the various time points, the viability of the microbes in the microdroplets does not need to be maintained after the microdroplets are prepared. This may have one or more advantages, for example, increased flexibility in the materials and manner used to prepare the microdroplets, and/or increased ease of handling the microdroplets (e.g., no need to maintain temperature, oxygen levels, etc.). In addition, because the viability of the microbes does not need to be maintained, the means used for assessing viability can be allowed to inhibit growth or kill the microbes, and additional tests that inhibit growth or kill the microbes can be conducted (e.g., lysing the microbes to conduct genetic analysis). It may be possible to maintain the viability of the microdroplets in Mode 4, so that the viability of microdroplets prepared after culturing for various times can also be assessed over time. In this way the features of Mode 4 can be combined with those of certain embodiments disclosed herein where the viability of microdroplets is assessed over time (e.g., embodiments of Mode 2).

In some embodiments, the method of measuring microbial viability includes incubating the one or more portions of samples contacted with antimicrobial for different periods of time prior to forming one or more populations of microdroplets, whereby microdroplets are formed from each of the one or more portions of samples at different time points. In some embodiments, the one or more portions of samples are incubated for a time period sufficient to allow any bacterial density-dependent phenomena to occur. In some embodiments, the one or more portions of samples are incubated over a period of any one or more of 0 hr, 0.1 hr, 0.2 hr, 0.5 hr, 1 hr, 2 hr, 3 hr, 4 hr, 5 hr, 6 hr, 7 hr, 8 hr, 9 hr, 10 hr, 11 hr, 12 hr, 15 hr, 18 hr, 20 hr, or 24 hr prior to forming microdroplets. In some embodiments, following formation of microdroplets, the microdroplets are measured to determine microbial viability. In some embodiments, the measurement of microdroplets may be performed irrespective of whether the microbes encapsulated within the microdroplets are viable or not viable. In some embodiments measuring microbial viability comprises obtaining a measure of microbial viability from a discrete subset of microdroplets from a first population of microdroplets from a first portion of the sample measured at a first time point, and obtaining a measure of microbial viability from a discrete subset of microdroplets from a second population of microdroplets from the first portion of the sample measured at a second time point. In some embodiments, the measure of microbial viability is whether an indicator of microbial viability exceeds a preset threshold. A subset of microdroplets includes one, or more than one microdroplet, for example, a subset includes, or includes at least, 1, 2, 3, 4, 5, 6, 7, 8, 9, 10, 20, 30, 40, 50, 60, 70, 80, 90, 100, 200, 300, 400, 500, 600, 700, 800, 900, 1000, 1500, 2000, 3000, 4000, 5000, 6000, 7000, 8000, 9000, or 10000 microdroplets, or a range defined by any two of the preceding values, for example 1-5, 1-10, 5-10, 5-20, 10-50, 10-100, 50-100, 50-500, 100-500, 100-1000, 500-1000, 500-5000, 1000-5000, 1000-10000, or 5000-10000 microdroplets. Additional details regarding the measure of microbial viability in the subsets of microdroplets that can be used in embodiments of Mode 4 are provided herein, including but limited to the disclosures regarding Modes 1, 2 and 3. In some embodiments, the method of measuring microbial viability using microdroplets formed at each time point provide advantages, including, for example, AST reads at an endpoint, allowing for detection chemistries that impact viability, bulk culturing of the sample prior to microdroplet generation, allowing for a determination of density-dependent resistant mechanisms, and maximized microdroplet occupancy.

In some embodiments, measuring microbial viability in droplets is performed using a technology that affects viability of the microorganism. A technology that affects viability of a microorganism may include, for example, a technology that uses lysis of the microorganism, such as determination of bacterial concentration by genetic analysis techniques, which may include, for example, qPCR or fluorescence in-situ hybridization (FISH) following bacterial lysis. In some embodiments, measuring microbial viability in droplets is performed using a technology that does not affect viability of the microorganism. A technology that does not affect viability of a microorganism may include, for example, measurement of solution turbidity, pH or fluorescence of a metabolically active dye.

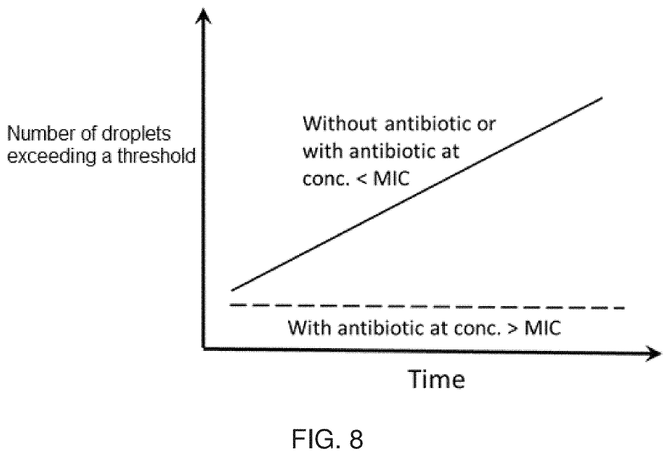

An embodiment of Mode 4 is depicted in FIG. 8, which shows microbial viability measured as a function of time in terms of fluorescence intensity. Microdroplets that are not exposed to antimicrobial or are exposed to antimicrobial below the MIC increase in fluorescence intensity (solid line). In contrast, microdroplets exposed to antimicrobial above a MIC do not exceed a preset threshold, and thus no increase in fluorescence intensity is observed (dashed line).

Time points for culturing portions of sample with antimicrobial prior to forming microdroplets are selected in the range from time 0 (e.g., the time at which antimicrobial is contacted with the portions of sample) to time 24 hours. Thus, in some embodiments, the time points include culturing for 0, 0.5, 1, 2, 3, 4, 5, 6, 7, 8, 9, 10, 11, 12, 13, 14 or 15 minutes, or 0.5, 0.75, 1, 1.5, 2, 2.5, 3, 3.5, 4, 4.5, 5, 5.5, 6, 7, 8, 9, 10, 12, 15, 18, 21, or 24 hours, or an amount of time within a range defined by any two of the aforementioned values. In some embodiments, the time points include culturing for 0 to 15 minutes, 0 to 10 minutes, 0 to 5 minutes, 0 to 1 minutes, 5 to 15 minutes, 5 to 10 minutes, or 10 to 15 minutes, or 0 to 24 hours, 0 to 21, 0 to 18, 0 to 15, 0 to 12, 0 to 10, 0 to 6, 0 to 5 hours, 0 to 4 hours, 0 to 3 hours, 0 to 2 hours, 0 to 1 hours, 0 to 0.5 hours, 0.25 to 6 hours, 0.25 to 5 hours, 0.25 to 4 hours, 0.25 to 3 hours, 0.25 to 2 hours, 0.25 to 1 hour, 0.25 to 0.5 hours, 1 to 6 hours, 1 to 5 hours, 1 to 3 hours, or 1 to 2 hours. Following culture for the given period of time, microdroplets are formed and a measure of microbial viability is determined, typically as soon as feasible after forming the microdroplet. A determination of microbial viability is made to determine whether the microbe is susceptible and/or not susceptible to an antimicrobial. In some embodiments, a determination of whether the microbe is susceptible and/or not susceptible to the antimicrobial is completed more quickly using the microdroplet susceptibility testing described herein than when testing antimicrobial susceptibility when not formed in microdroplets.

Some embodiments of Mode 4 include measuring microbial viability of microdroplets by forming microdroplets at each time point and determining whether microbial viability exceeds a predetermined threshold for each population of microdroplets formed at each measurement point. FIG. 7 illustrates a schematic for Mode 4. The sample is divided into portions, and each portion is contacted with antimicrobial at different concentrations spanning a desired range. For each portion, microdroplets are prepared at each desired measurement time point. In some embodiments, a discrete subset of microdroplets is measured at each time point, optionally after an additional period of culturing the portions of sample as disclosed herein, and microbial viability is measured on a discrete subset of microdroplets (such as in Mode 1), or on the same subset of microdroplets (such as in Mode 2). In some embodiments, a measure of microbial viability is assessed when a signal of microbial viability exceeds a threshold (such as in Mode 3). However, rather than forming microdroplets at time 0, and subsequently measuring microbial viability of the microdroplets at subsequent time points, the microdroplets are formed only at the measurement time points, such that the portion of sample is incubated with the antimicrobial until the measurement time point, at which point microdroplets are formed and measured. In some embodiments, measurement can be performed in any of the methods disclosed herein, including, for example, using a high throughput microdroplet analyzer or an indexed array. In some embodiments, number or percentage of microdroplets exceeding the threshold is determined as a function of time for each concentration of antimicrobial, and microbial viability and MIC is assessed from the measurements.

Although in each of the modes described above, the steps of each is described as a discrete process, one or more of these steps may be performed in a system. Thus, for example, one or more of the processes may be performed in a microfluidic device. A microfluidic device may be used to automate the process and/or allow concomitant processing of multiple samples. One of skill in the art is well aware of methods in the art for collecting, handling, and processing biological fluids that can be used in the practice of the present disclosure. Additionally, the microfluidic devices for the various steps can be combined into one system for carrying out any of the modes described herein.

### Measurement of Microbial Viability

As described, some embodiments provided herein relate to methods for antimicrobial susceptibility testing (AST). Any of the methods, embodiments, systems or modes described herein may be interchangeable, modified, or varied in such a way to allow for microbial AST by encapsulating microbes within microdroplets. Without wishing to be bound by theory, embodiments of the methods, embodiments, systems and modes described herein may have one or more advantages for measuring microbial viability, including a high sensitivity near the MIC, rapid determination of microbial viability, and/or bulk determination of microbial viability over a broad range of antimicrobial concentrations.

In any of the embodiments, methods, systems or modes described herein, microdroplets may be incubated in different antimicrobial concentrations for any period of time. The microbial growth may be monitored during the incubation period and the incubation period can continue until there is a sufficient difference in detection signal, e.g., fluorescence or microbial counts, between microdroplets. For example, in some embodiments, incubation can be for about 15, 30, or 45 seconds, for about 1, 2, 3, 5, 10, 15, 20, 25, 30, 35, 40, 45, 50, 55, 60, 90, 120, 150, 180, 210, 240, 300, 360 minutes or more. In one embodiment, the incubation is more than 2 hours, e.g., at least about 2 hours, at least about 6 hours, at least about 12 hours, at least about 24 hours, at least about 2 days, at least about 3 days. Depending on the proliferation and/or growth rate of encapsulated microbes, one of skill in the art can determine optimum incubation duration for subsequent analysis, e.g., cell viability analysis.

In any of the embodiments, methods, systems or modes described herein, a discrete subset of microdroplets having a microbe encapsulated therein may be measured to determine the susceptibility of the microbe within the microdroplet to antimicrobials. In some embodiments, determining the susceptibility of a microbe to an antimicrobial is performed by measuring a fluorescence intensity of a microdroplet. In some embodiments, determining the susceptibility of a microbe to an antimicrobial is performed during incubation of the microbes or after incubation of the microbes. In some embodiments, determining the susceptibility of the microbe to the antimicrobial is performed continuously by continually monitoring microbial viability, or periodically by monitoring microbial viability at one or more distinct time points.

In any of the embodiments, methods, systems or modes described herein, a sample may be divided into portions of sample, and each portion of sample may be contacted with a different concentration of antimicrobial. In some embodiments, for each portion of sample, a measure of microbial viability is measured for a discrete subset of microdroplets as a function of time by either placing and interrogating them on an indexed array or by using a high throughput microdroplet reader. Measurement can take place, for example, by measuring a fluorescent intensity of the discrete subset of microdroplets at an initial time point (e.g. first time point) and at subsequent time points (e.g., second, third, fourth, etc.). A measured fluorescence intensity for the discrete subset of microdroplets may be used to assess bacterial viability at each antimicrobial concentration. In some embodiments, measurement is performed at multiple time points during incubation, for example, at a first time point and then at a second time point. In some embodiments, the measurement of microbial viability on a discrete subset of microdroplets (e.g., a single microdroplet) is measured at time 0, 1 hour, 2 hours, 3 hours, or more time points. In some embodiments, a measurement is performed at any number of desired time points within a time frame from time 0 to time 24 hours. As disclosed herein, time points may be selected at various frequencies and over ranges of time. Furthermore, as disclosed herein, time points are selected to allow sufficient time for a determination that the microbe is susceptible and/or not susceptible to an antibiotic, for example, when the signal of microbial viability exceeds a threshold. In some embodiments, a determination of whether the microbe is susceptible and/or not susceptible to the antimicrobial is completed more quickly using the microdroplet susceptibility testing described herein than when testing antimicrobial susceptibility when not formed in microdroplets. Thus, in some embodiments, determining susceptibility of the microbes to the antimicrobial is completed in a time within a range of 3-24 hours, 3-20 hours, 3-15 hours, 3-8 hours, 5-20 hours, 5-15 hours, or 5-8 hours, or within an amount of time within a range defined by any two of the aforementioned values. In some embodiments determining susceptibility of the microbes to the antimicrobial is completed in not more than 24 hours; in not more than 15 hours; in not more than 12 hours; in not more than 10 hours; in not more than 8 hours; in not more than 5 hours; in not more than 3 hours)

In some embodiments, the time points at which the measurement of microbial viability is selected in the range from time 0 (e.g., the time at which the microdroplets encapsulating microbes are formed) to time 24 hours. Thus, in some embodiments, the time points include a measurement at 0, 0.5, 1, 2, 3, 4, 5, 6, 7, 8, 9, 10, 11, 12, 13, 14 or 15 minutes or 0.5, 0.75, 1, 1.5, 2, 2.5, 3, 3.5, 4, 4.5, 5, 5.5, 6, 7, 8, 9, 10, 12, 15, 18, 21, or 24 hours, or an amount of time within a range defined by any two of the aforementioned values. In some embodiments, the time points include measurement at any given frequency from 0 to 15 minutes, 0 to 10 minutes, 0 to 5 minutes, 0 to 1 minutes, 5 to 15 minutes, 5 to 10 minutes, or 10 to 15 minutes, 0 to 24 hours, 0 to 21, 0 to 18, 0 to 15, 0 to 12, 0 to 10, 0 to 6, 0 to 5 hours, 0 to 4 hours, 0 to 3 hours, 0 to 2 hours, 0 to 1 hours, 0 to 0.5 hours, 0.25 to 6 hours, 0.25 to 5 hours, 0.25 to 4 hours, 0.25 to 3 hours, 0.25 to 2 hours, 0.25 to 1 hour, 0.25 to 0.5 hours, 1 to 6 hours, 1 to 5 hours, 1 to 3 hours, or 1 to 2 hours, or until a determination of antimicrobial susceptibility can be realized. In some embodiments, antimicrobial susceptibility is made from a time of contacting a sample with an antimicrobial to making a determination of antimicrobial susceptibility in no more than 24, 20, 15, 10, 8, 5, or 3 hours, or an amount of time sufficient to make a determination that the microbe is susceptible and/or not susceptible to the tested antimicrobial.

In any of the embodiments, methods, systems or modes described herein, a measure of microbial viability may be obtained from a discrete subset of microdroplets from a first portion exposed to a first concentration of antimicrobial at a first time point, and a measure of microbial viability is obtained from a discrete subset of microdroplets from the first portion exposed to the first concentration of antimicrobial at a second time point. In some embodiments, an average measurement of microbial viability from a discrete subset of microdroplets at the first time point is compared to an average measurement of microbial viability from a discrete subset of microdroplets measured at a second time point. Microbes can be observed, for example, for growth in the presence of the antimicrobials (to determine the resistance of the bacteria to the particular antimicrobials), cell death (to determine bactericidal activity), and/or inhibition of growth (to determine bacteriostatic activity). For example, microbe growth and/or cell death can be assessed by: (i) counting the number of microbes in a subset of microdroplets as compared to a control or reference; (ii) total amount of microbes in the subset of microdroplets, as compared to a control or reference; (iii) ratio of cells expressing at least one microbe marker in the subset of microdroplets, as compared to a control or reference; (iv) relative metabolite levels in the subset of microdroplets, as compared to a control or reference; or (v) any combinations thereof. In some embodiments, microbial growth or a functional response of microbes can be determined or monitored in real-time, e.g., by microscopy or flow cytometry.

In any method known in the art for determining the viability of microbes in a sample can be used for determining viability of microbes encapsulated within microdroplets over time and compared to the growth of encapsulated microbes exposed to different concentrations of antimicrobial. Generally, cell viability can be assayed using cytolysis or membrane leakage assays (such as lactate dehydrogenase assays), mitochondrial activity or caspase assays (such as Resazurin and Formazan (MTT/XTT) assays), production of reactive oxygen species (ROS) assays, functional assays, or genomic and proteomic assays. Exemplary methods include, but are not limited to, ATP test, ROS test, Calcein AM, pH sensitive dyes, Clonogenic assay, Ethidium homodimer assay, Evans blue, Fluorescein diacetate hydrolysis/Propidium iodide staining (FDA/PI staining), Flow cytometry, Formazan-based assays (MTT/XTT), Green fluorescent protein, Lactate dehydrogenase (LDH), Methyl violet, Propidium iodide, DNA stain that can differentiate necrotic, apoptotic and normal cells, Resazurin, Trypan Blue (a living-cell exclusion dye (dye only crosses cell membranes of dead cells)), 7-aminoactinomycin D, TUNEL assay, cell labeling or staining (e.g., a cell-permeable dye (e.g., Carboxylic Acid Diacetate, Succinimidyl Ester (Carboxy-DFFDA, SE)), a cell-impermeable dye, cyanine, phenantridines, acridines, indoles, imidazoles, a nucleic acid stain, a cell permeant reactive tracer (e.g., intracellularly-activated fluorescent dyes CMRA, CMF2HC (4-Chloromethyl-6,8-Difluoro-7-Hydroxycoumarin), CMFDA (5-Chloromethylfluorescein Diacetate), CMTMR (5-(and -6)-(((4-Chloromethyl)Benzoyl)Amino)Tetramethylrhodamine), CMAC (7-Amino-4-Chloromethylcoumarin), CMHC (4-Chloromethyl-7-Hydroxycoumarin)) or any combinations thereof), fluorescent DNA dyes (e.g., DAPI, Heochst family, SYBR family, SYTO family (e.g., SYTO 9), SYTOX family (e.g., SYTOX green), ethidium bromide, propidium iodide, acridines, or any combinations thereof); chromogenic dyes (e.g., eosin, hematoxilin, methylene blue, azure, or any combinations thereof); cytoplasm stain (e.g., calcofluor white, periodic acid-Schiff stain, or any combinations thereof); metabolic stains (e.g., any metabolic stains described herein, any diacetate dye (including, rhodamine based-dye, fluorescin, or any combinations thereof), resazurin/resorufin (alamar blue); ROS stains (e.g., any ROS stains described herein, DCFDA and related family, calcein-acetoxymethyl and related family); membrane stains (e.g., bodipy, FM 1-43, FM 4-64, and functionally equivalent thereof, CellMask™ stains, Dil, DiO, DiA); biologic stains (e.g., labeled antibodies, labeled chitin-binding protein), optical imaging, microscopic imaging after staining, ELISA, mass spectrometric analysis (e.g., of peptides, proteins, glycopeptides, lipopeptides, carbohydrates, and/or metabolites), modification of metabolomic fingerprint, degradation of RNA or of protein content and the like. In some embodiments, the detection of the growth or functional response of the microbe to the antimicrobial can be done using solid phase, microfluidics or microdroplet based assays. In some embodiments, the detection of the growth or functional response of the microbe to the antimicrobial can comprise use of a mass spectrometer. In some embodiments, the detection of the growth or functional response of the microbe to the antimicrobial can comprise detection of at least one metabolite, or a metabolic profile. In some embodiments, the detection of the growth or functional response of the microbe to the antimicrobial can comprise detection of transcriptional changes. In some embodiments, microbial viability in microdroplets may be determined by performing imaging of microbes in microdroplets to collect both proliferation and morphology data. In some embodiments, methods of analysis disclosed herein may be applied to microbial count data to determine MIC. In some embodiments, a measure of microbial viability includes distributing a discrete subset of microdroplets on an indexed array, or by using a rapid scanner.

In any of the embodiments, methods, systems or modes described herein, a measure of microbial viability may be performed by measuring a fluorescent dye in the microdroplets. In some embodiments, the fluorescent dye is a viability indicator dye. In some embodiments, the fluorescent dye is resazurin. Resazurin is reduced to resorufin as a result of bacterial proliferation. Resorufin has a high fluorescence quantum yield compared to resazurin, resulting in increased fluorescence intensity as bacteria grow within microdroplets. In some embodiments, the measure of microbial viability may be assessed and the MIC may be determined from measurements of microdroplet fluorescence. In some embodiments, an algorithm is developed and used for MIC determination.

### Microdroplets

Microdroplets provided herein, and prepared in any of the modes described herein for measuring microbial viability may be manufactured using a microfluidic device or other device for microdroplet generation. In some embodiments, the microdroplets are of sufficient size to encapsulate a microbe of interest. For example, the microdroplets may range in size from 2 μm to about 500 μm, such as, for example, 2, 3, 4, 5, 6, 7, 8, 9, 10, 15, 20, 25, 30, 35, 40, 45, 50, 60, 70, 80, 90, 100, 110, 120, 130, 10, 150, 160, 170, 180, 190, 200, 250, 300, 350, 400, 450, or 500 μm in diameter, or a diameter within a range defined by any two of the aforementioned values. In some embodiments, microdroplets are in a range from 2 μm to 500 μm, 2 μm to 50 μm, 2 μm to 10 μm, 10 μm to 200 μm, 10 μm to 50 μm, 50 μm to 200 μm, or 50 μm to 100 μm. The size of the microdroplets may be based on the mode of preparation of the microdroplets or the specific desired size range. One of skill in the art will appreciate that the microdroplet size can be modified in size to accommodate the specific microbe of interest or the particular assay being used. In some embodiments, the microdroplets have a volume in the range of picoliters, for example 0.001, 0.01, 0.1, 1, 2, 3, 4, 5, 6, 7, 8, 9, 10, 20, 30, 40, 50, 60, 70, 80, 90, 100, 200, 300, 400, 500, 600, 700, 800, 900, or 1000 pl, or a volume within a range defined by any two of the aforementioned values. In some embodiments, microdroplets have a volume of 0.001 pl to 1000 pl, 0.001 pl to 100 pl, 100 pl to 1000 pl, 100 pl to 500 pl, or 500 pl to 1000 pl.

In any of the embodiments, methods, systems, or modes described herein, microdroplets may be formed after contacting a sample with an antimicrobial. In some embodiments, microdroplets are formed within 1, 2, 3, 4, 5, 10, 15, 30, 45, or 60 seconds, or within 1, 2, 3, 4, 5, 10, 15, 20, 25, 30, 35, 40, 45, 50, 55, or 60 minutes, or within 1, 2, 3, 4, 5, 6, 8, 10, 12, 14, 16, 18, 20, 22, or 24 hours after contacting a sample with antibiotics, or within a time frame defined by any two of the aforementioned values. In some embodiments, the microdroplets are formed within 1 second, 30 seconds, 1 minute, 15 minutes, 30 minutes, 1 hour, or 2 hours of contacting the one or more portions of samples with the antimicrobial.

In any of the embodiments, methods, systems or modes described herein, the microdroplets may be stable water-in-oil emulsions created by dispersing a sample containing a microbe of interest in a continuous hydrophobic oil phase containing a surfactant. Examples of surfactants may include, but are not limited to, sulfonates, alkyl sulfates, monoesters of polyalkoxylated sorbitan, a polyester polyols, aliphatic alcohol esters, aromatic alcohol esters, tall oil fatty acid diethanolamide, polyoxyethylene (5) sorbitan monooleate, ammonium salts of polyacrylic acid, ammonium salts of a 2-acrylamido-2-methylpropane sulfonic acid/acrylic acid copolymer, alkylsulfonate, alkylarylsulfonate, α-olefin sulfonate, diphenyl ether sulfonate, sorbitan monooleate, α-sulfo fatty acid methyl ester. Combinations of surfactants also may be used.

Other methods of microdroplet production may include, for example, membrane emulsification, external forces, such as mechanical shear (impeller driven microdroplet generators), or electrical forces, such as dielectrophoresis modulated microdroplet generators.

In any of the embodiments, methods, systems or modes described herein, microdroplet size, surfactant, and oil may be optimized to reduce or eliminate diffusion of molecules (e.g. assay reagents, dyes, antimicrobials, nutrients, metabolites) from microdroplet-to-droplet or from microdroplet-to-oil. In some embodiments, microdroplet size, surfactant, and oil is optimized to enable or enhance gas diffusion from the oil phase into microdroplets. In some embodiments, microdroplet size, surfactant, and oil is optimized to improve microdroplet stability and reduce undesired microdroplet merging or fusion. In some embodiments, microdroplet composition is optimized to facilitate AST reactions occurring in clinical specimen matrices.

In any of the embodiments, methods, systems or modes described herein, the production of the microdroplet may be performed in the presence of a sample containing a microbe. In some embodiments, the production of a microdroplet in the presence of a microbe results in encapsulation of a single microbe within a microdroplet. Occupancy and distribution of microbes within microdroplets can be varied by adjusting the concentration of microbes in the sample. A pre-determined concentration of an antimicrobial and viability indicator dye can also be incorporated into the microdroplets at the point where microdroplets are formed or can be added to each microdroplet at a later time point using approaches such as micro-injection or microdroplet merging. These microdroplets can then be collected in a vial, incubated at appropriate bacterial growth conditions, and monitored at regular intervals.

Methods disclosed herein for performing AST using microdroplets can be applied to any embodiment or instrument capable of measuring a microdroplet property of interest over time. These methods, while independent of embodiments used to generate and interrogate microdroplets, may benefit from certain microdroplet characteristics. Some of these characteristics are size/volume of microdroplets, stability of microdroplets over the duration of AST, and composition of oil and aqueous systems that supports bacterial proliferation. Systems that one can use for monitoring fluorescence intensity of microdroplets include: (a) an indexed array for the placement of microdroplets and their subsequent fluorescence readout or (b) an instrument capable of high throughput interrogation of microdroplets.

In any of the embodiments, methods, systems or modes described herein, a sample may be divided into portions, and each portion may be exposed to a different concentration of antimicrobial. In some embodiments, the microbe in the sample is first encapsulated within a microdroplet and then exposed to an antimicrobial. In some embodiments, the microbe is encapsulated within a microdroplet concomitantly with exposure to an antimicrobial. Thus, the sample containing a microbe may be exposed to an antimicrobial before, during, or after encapsulation within microdroplets. Without limitations, the microbes in one or more portions can be incubated with at least one antimicrobial agent, including two, three, four or more antimicrobial agents, e.g., to determine the efficacy of a combination therapy. At least one (e.g., one, two, three, four, five, six, seven, eight, nine, ten, or more) of the portions can be incubated without addition of any antimicrobial agents for serving as a control. Alternatively, or in addition, at least one (e.g., one, two, three, four, five, six, seven, eight, nine, ten, or more) of the portions can be incubated with a broad-spectrum antimicrobial agent for serving as a positive control.

In any of the embodiments, methods, systems, or modes described herein, each portion of sample with different concentration of antimicrobial may be introduced to a microdroplet generator, and microdroplets are generated to encapsulate a single microbe, or an average of less than one microbe per microdroplet. In some embodiments, the microdroplets are generated concomitantly with exposure to an antimicrobial. In some embodiments, the microdroplets are generated at time zero from a sample with added antimicrobial at different concentrations spanning a desired clinical range. In some embodiments, the microdroplets are measured to determine microbial viability. Measuring a microdroplet may include measuring a subset of microdroplets. A subset of microdroplets includes one, or more than one microdroplet, for example, a subset includes, or includes at least, 1, 2, 3, 4, 5, 6, 7, 8, 9, 10, 20, 30, 40, 50, 60, 70, 80, 90, 100, 200, 300, 400, 500, 600, 700, 800, 900, 1000, 1500, 2000, 3000, 4000, 5000, 6000, 7000, 8000, 9000, or 10000 microdroplets, or a range defined by any two of the preceding values, for example 1-5, 1-10, 5-10, 5-20, 10-50, 10-100, 50-100, 50-500, 100-500, 100-1000, 500-1000, 500-5000, 1000-5000, 1000-10000, or 5000-10000 microdroplets.

The microdroplets encapsulating microbes with various concentrations of antimicrobial can be incubated under any conditions suitable for microbial growth. One of skill in the art can readily determine optimum culture conditions for microbial growth (e.g., bacterial growth), e.g., incubating them at a suitable temperature and atmosphere such as at from about 20° C. to about 45° C. in the presence or absence of adequate levels of oxygen and/or carbon dioxide. In some embodiments, incubation is at from about 25° C. to about 40° C., or from about 30° C. to about 42° C., or from about 35° C. to about 40° C. In one embodiment, incubation is at about 37° C.

### Antimicrobial Agents

Any of the embodiments, methods, systems or modes described herein, may include contacting a sample with an antimicrobial agent. In any of the embodiments or modes described herein, an antimicrobial agent may be incorporated into a microdroplet or the antimicrobial agent can be exposed to a microdroplet. In some embodiments, the antimicrobial agent is dried in the device at a specified concentration and/or amount and is reconstituted by a portion of the sample containing microbes before forming droplets. The amount of the dried antimicrobial can be adjusted such that when reconstituted by the portion of sample, the resultant concentration falls within a desired range. In some embodiments, the antimicrobial agent is added to the microdroplets after they are prepared, rather than to the sample prior to formation of the microdroplets.

In any of the embodiments, methods, systems or modes described herein, an antimicrobial agent may be an agent that is naturally occurring, semisynthetic, or fully synthetic agents that inhibit the growth of microbes (e.g., bacteria, fungi, viruses, parasites and microbial spores) thereby preventing their development and microbial or pathogenic action. Antimicrobial agents are known to those of skill in the art. However, by way of example, an antimicrobial agent can be selected from the group consisting of small organic or inorganic molecules; saccharines; oligosaccharides; polysaccharides; biological macromolecules, e.g., peptides, proteins, and peptide analogs and derivatives; peptidomimetics; antibodies and antigen binding fragments thereof; nucleic acids; nucleic acid analogs and derivatives; glycogens or other sugars; immunogens; antigens; an extract made from biological materials such as bacteria, plants, fungi, or animal cells; animal tissues; naturally occurring or synthetic compositions; and any combinations thereof. In some embodiments, an antimicrobial agent includes antibacterial agents (or antibiotic), antifungal agents, antiprotozoal agents, antiviral agents and mixtures thereof.

Non-limiting examples of antibiotics include, for example, an aminoglycoside (including, for example, amikacin, gentamicin, kanamycin, neomycin, netilmicin, tobramycin, paromomycin, streptomycin, spectinomycin), an ansamycin (including, for example, geldanamycin, herbimycin, rifaximin), a carbacephem (including, for example, loracarbef), a carbapenem (including, for example, ertapenem, antipseudomonal, doripenem, imipenem, cilastatin, meropenem, biapenem, panipenem), a cephalosporin (including for example, cefazolin, cefalexin, cefadroxil, cefapirin, cefazedone, cefazaful, cefradine, cefroxadine, ceftezole, cefaloglycin, cefacetrile, cefalonium, cefaloridine, cefalotin, cefatrizine, cefaclor, cefotetan, cephamycin, cefoxitin, cefprozil, cefuroxime, cefuroxime axetil, cefamandole, cefminox, cefonicid, ceforanide, cefotiam, cefbuperazone, cefuzonam, cefmetazole, carbacephem, loracarbef, cefixime, ceftriaxone, antipseudomonal, ceftazidime, cefoperazone, cefdinir, cefcapene, cefdaloxime, ceftizoxime, cefmenoxime, cefotaxime, cefpiramide, cefpodoxime, ceftibuten, defditoren, cefetamet, cefodizime, cefpimizole, cefsulodin, cefteram, ceftiolene, oxacephem, flomoxef, latamoxef, cefepime, cefozopran, cefpirome, cefquinome, ceftaroline fosamil, ceftolozane, ceftobiprole, ceftiofur, cefquinome, cefovecin), a glycopeptide (including, for example, vancomycin, oritavancin, telavancin, teicoplanin, dalbavancin, ramoplanin), a lincosamide (including, for example, clindamycin, lincomycin), a lipopeptide (including, for example, daptomycin), a macrolide (including, for example, azithromycin, clarithromycin, erythromycin, roxithromycin, telithromycin, spiramycin), a monobactam (including, for example, aztreonam, tigemonam, carumonam, nocardicin A), a nitrofuran (including, for example, furazolidone, nitrofurantoin), an oxazolidonone (including, for example, linezolid, posizolid, radezolid, torezolid), a penicillin (including for example, a penam, a β-lactam, benzylpenicillin, benzathine benzylpenicillin, procaine benzylpenicillin, phenoxymethylpenicillin, propicillin, pheneticillin, azidocillin, clometocillin, penamecillin, cloxacillin, dicloxacillin, flucloxacillin, oxacillin, nafcillin, methicillin, amoxicillin, ampicillin, pivampicillin, hetacillin, bacampicillin, metampicillin, talampicillin, epicillin, ticarcillin, carbenicillin, carindacillin, temocillin, piperacillin, azlocillin, mezlocillin, mecillinam, pivmecillinam, sulbenicillin), a penem (including, for example, faropenem or ritipenem), a polypeptide (including, for example, bacitracin, colistin, polymyxin B), a quinolone (including, for example, ciprofloxacin, enocaxin, gatifloxacin, gemifloxacin, levofloxacin, lomefloxacin, moxifloxacin, nadifloxacin, nalidixic acid, norfloxacin, ofloxacin, trovafloxacin, grepafloxacin, sparfloxacin, temafloxacin), a sulfonamide (including, for example, mafenide, sulfacetamide, sulfadiazine, silver sulfadiazine, sulfadimethoxine, sulfamethizole, sulfamethoxazole, sulfanilimde, sulfasalazine, sulfisoxazole, trimethoprim-sulfamethoxazole, co-trimoxazole, sulfonamidochrysoidine), a tetracycline (including, for example, demeclocycline, doxycycline, metacycline, minocycline, oxytetracycline, tetracycline), or other antibiotic, including, for example, acrosoxacin, amifloxacin, amikacin, amoxycillin, ampicillin, arsphenamine, aspoxicillin, azidocillin, azithromycin, aztreonam, balofloxacin, biapenem, brodimoprim, capreomycin, cefaclor, cefadroxil, cefatrizine, cefcapene, cefdinir, cefetamet, cefoxitin, cefprozil, cefroxadine, ceftarolin, ceftazidime, ceftibuten, ceftmetazole, ceftobiprole, cefuroxime, cephalexin, cephalonium, cephaloridine, cephamandole, cephazolin, cephradine, chloramphenicol, chlorquinaldol, chlortetracycline, ciclacillin, cinoxacin, ciprofloxacin, clarithromycin, clavulanic acid, clindamycin, clofazimine, clofazimine, cloxacillin, colistin, cycloserine, dalfopristin, danofloxacin, dapsone, daptomycin, demeclocycline, dicloxacillin, difloxacin, doripenem, doxycycline, enoxacin, enrofloxacin, erythromycin, ethambutol, ethionamide, fleroxacin, flomoxef, flucloxacillin, flumequine, fosfomycin, fusidic acid, gentamycin, imipenem, isoniazid, kanamycin, lc benzylpenicillin, levofloxacin, linezolid, mandelic acid, mecillinam, meropenem, metronidazole, minocycline, moxalactam, mupirocin, nadifloxacin, nalidixic acid, netilmycin, netromycin, nifuirtoinol, nitrofurantoin, nitroxoline, norfloxacin, ofloxacin, oxytetracycline, panipenem, pefloxacin, phenoxymethylpenicillin, pipemidic acid, piromidic acid, pivampicillin, pivmecillinam, platensimycin, prulifloxacin, pyrazinamide, quinupristin, rifabutin, rifampicin, rifapentine, rufloxacin, sparfloxacin, streptomycin, sulbactam, sulfabenzamide, sulfacytine, sulfametopyrazine, sulphacetamide, sulphadiazine, sulphadimidine, sulphamethizole, sulphamethoxazole, sulphanilamide, sulphasomidine, sulphathiazole, teicoplanin, teixobactin, temafioxacin, tetracycline, tetroxoprim, thiamphenicol, tigecyclin, tinidazole, tobramycin, tosufloxacin, trimethoprim, or vancomycin, and pharmaceutically acceptable salts or esters thereof.

Exemplary antifungal agents include, but are not limited to, 5-Flucytosin, Aminocandin, Amphotericin B, Anidulafungin, Bifonazole, Butoconazole, Caspofungin, Chlordantoin, Chlorphenesin, Ciclopirox Olamine, Clotrimazole, Eberconazole, Econazole, Fluconazole, Flutrimazole, Isavuconazole, Isoconazole, Itraconazole, Ketoconazole, Micafungin, Miconazole, Nifuroxime, Posaconazole, Ravuconazole, Tioconazole, Terconazole, Undecenoic Acid, and pharmaceutically acceptable salts or esters thereof.

Exemplary antiprotozoal agents include, but are not limited to, Acetarsol, Azanidazole, Chloroquine, Metronidazole, Nifuratel, Nimorazole, Omidazole, Propenidazole, Secnidazole, Sinefungin, Tenonitrozole, Temidazole, Tinidazole, and pharmaceutically acceptable salts or esters thereof.

Exemplary antiviral agents include, but are not limited to, Acyclovir, Brivudine, Cidofovir, Curcumin, Desciclovir, 1-Docosanol, Edoxudine, gQ Fameyclovir, Fiacitabine, Ibacitabine, Imiquimod, Lamivudine, Penciclovir, Valacyclovir, Valganciclovir, and pharmaceutically acceptable salts or esters thereof.

In any of the embodiments, methods, systems or modes described herein, the antimicrobial agent may be selected from the group consisting of amoxicillin/clavulanate, amikacin, ampicillin, aztreonam, ceftrazidime, cephalothin, chloramphenicol, ciprofloxacin, clindamycin, ceftriaxone, cefotaxime, cefuroxime, erythromycin, cefepime, gentamicin, imipenem, levofloxacin, linezolid, meropenem, minocycline, nitrofurantoin, oxacillin, penicillin, piperacillin, ampicillin/sulbactam, trimethoprim/sulfamethoxazole or co-trimoxazole, tetracycline, tobramycin, vancomycin, or any combinations thereof.

In any of the embodiments, methods, systems or modes described herein, a newly developed antimicrobial agent may be incorporated into the microdroplet or exposed to the microdroplet in order to determine the efficacy of the newly developed antimicrobial agent, or for a determination of the susceptibility of the microbe to the newly developed antimicrobial agent.

In any of the embodiments, methods, systems or modes described herein, the concentration of an antimicrobial agent in AST provided herein may be provided in various concentrations in order to determine the susceptibility range. In some embodiments, the concentration of antimicrobial is provided in various concentrations in order to determine a minimum inhibitory concentration (MIC). The broad range of antimicrobials provided herein may be provided at various concentrations and have various efficacies. Thus, the concentration of antimicrobial will vary depending on the selected antimicrobial. The MIC of any given antimicrobial is known to those of skill in the art, including the range of any given antimicrobial that may be useful for antimicrobial susceptibility testing. The concentrations of antimicrobial are selected to include a range of concentrations of antimicrobial that includes a concentration of antimicrobial that is or is suspected of being the minimum inhibitory concentration (MIC), as described in more detail herein. In some other embodiments, the concentration range of antimicrobial covers the clinically or physiologically relevant concentration range but may not include concentrations that will enable determination of MIC. In some embodiments, one or more of the portions of sample is not exposed to any antimicrobial (antimicrobial concentration of zero), e.g. for purposes of a control. Thus, for example, where a microdroplet is exposed to an antimicrobial (or where an antimicrobial is incorporated into the microdroplet upon formation of the microdroplet) a concentration of antimicrobial may range from about 0.001 μg/ml to about 5000 μg/ml, such as 0.001, 0.01, 0.1, 1, 10, 100, 1000, or 5000 μg/ml, or a range within an amount defined by any of the aforementioned values. In some embodiments, the antimicrobial concentration is from 0.001 μg/ml to 5000 μg/ml, 0.001 μg/ml to 1000 μg/ml, 0.001 μg/ml to 100 μg/ml, 0.001 μg/ml to 10 μg/ml, 10 μg/ml to 5000 μg/ml, 10 μg/ml to 1000 μg/ml, 10 μg/ml to 100 μg/ml, 100 μg/ml to 5000 μg/ml, 100 μg/ml to 1000 μg/ml, or 1000 μg/ml to 5000 μg/ml. In some embodiments, antimicrobial concentration is a serial dilution in order to determine the MIC. The serial dilution may be a dilution in an amount of 1, 2, 3, 4, 5, 6, 7, 8, 9, 10, 15, 20, 25, 30, 35, 40, 45, 50, 60, 70, 80, 90, 100, 150, 200, 300, 400, 500, 1000, or 10000-fold dilution, or a dilution within a range defined by any of the aforementioned values. In some embodiments, the dilution is in an amount of 1 to 10000 fold, 1 to 1000 fold, 1 to 100 fold, 1 to 10 fold, 10 to 10000 fold, 10 to 1000 fold, 10 to 100 fold, 100 to 10000 fold, or 100 to 1000 fold. In some embodiments, the concentration of antimicrobial is zero (e.g., no antimicrobial is present), thereby providing a control indicating normal microbial growth in the absence of any antimicrobial.

In some embodiments, a sample of microdroplets encapsulating a microbe is divided into one or more portions of microdroplets, and a first portion may be exposed to a first concentration of antimicrobial, a second portion may be exposed to a second concentration of antimicrobial, a third portion may be exposed to a third concentration of antimicrobial, and so forth for a desired number of portions exposed to a desired number of antimicrobial concentrations. Thus, for example, in a sample of microdroplets, a first concentration of antimicrobial exposed to a first portion of microdroplets may be 0.001 μg/ml, a second concentration of antimicrobial exposed to a second portion of microdroplets may be 0.005 μg/ml, and a third concentration of antimicrobial exposed to a third portion of microdroplets may be 0.01 μg/ml, and so forth for a desired number of portions exposed to a desired number of different concentrations of a particular antimicrobial or antimicrobials. In some embodiments, each portion may be exposed to a serial dilution of antimicrobial for a desired number of dilutions of antimicrobial. The range of concentrations and the number of portions of microdroplets may be ascertained based on the antimicrobial being tested, its clinically or physiologically relevant concentration range, the suspected microbe, or the particular assay being carried out, for example to cover a range of concentrations of antimicrobial that include a minimum inhibitory concentration of the antimicrobial.

### Microbes

Embodiments provided herein relate to measuring microbial viability. In any of the embodiments, methods, systems or modes described herein, a microbe may be encapsulated within a microdroplet during formation of the microdroplet. For example, in some embodiments, a sample containing a microbe is dispersed in a continuous hydrophobic oil phase containing a surfactant in conditions to generate water-in-oil microdroplets. In some embodiments, the sample containing a microbe is a clinical sample that has been processed. In some embodiments, the sample comprises one or more of peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen (including prostatic fluid), Cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates or other lavage fluids, blastocoel cavity, umbilical cord blood, or maternal circulation, which may be of fetal or maternal origin. In some embodiments, the sample is collected from a human, one or more companion animals, or one or more commercially important animals. In some embodiments, the human, one or more companion animals, or one or more commercially important animals has a microbial infection, such as a bacterial infection.

In any of the embodiments, methods, systems or modes described herein the sample may be a clinical sample, which may be obtained from a human subject, and may be processed to isolate a microbial population of interest from one or more components of the sample. The sample may be obtained, for example, as peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen (including prostatic fluid), Cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates or other lavage fluids, blastocoel cavity, umbilical cord blood, or maternal circulation, which may be of fetal or maternal origin.

In any of the embodiments, methods, systems or modes described herein, the sample may be a fluid or specimen obtained from an environmental source. For example, the fluid or specimen obtained from the environmental source can be obtained or derived from food products, food produce, poultry, meat, fish, beverages, dairy product, water (including wastewater), ponds, rivers, reservoirs, swimming pools, soils, food processing and/or packaging plants, agricultural places, hydrocultures (including hydroponic food farms), pharmaceutical manufacturing plants, animal colony facilities, or any combinations thereof. In some embodiments, the sample is a fluid or specimen collected or derived from a cell culture or from a microbe colony.

In any of the embodiments, methods, systems or modes described herein, it may be necessary or desired to process a sample prior to microbial encapsulation in the microdroplets. Even in cases where processing is not necessary, processing optionally can be done for convenience (e.g., as part of a regimen on a commercial platform). A processing reagent can be any reagent appropriate for use with the methods described herein. The sample processing step may include, for example adding one or more reagents to the sample. This processing can serve a number of different purposes, including, but not limited to, hemolyzing cells such as blood cells, dilution of sample, etc. The processing reagents may include, but are not limited to, surfactants and detergents, salts, cell lysing reagents, anticoagulants, degradative enzymes (e.g., proteases, lipases, nucleases, lipase, collagenase, cellulases, amylases and the like), and solvents, such as buffer solutions. In some embodiments, a processing reagent is a surfactant or a detergent. In some embodiments, the sample is a clinical sample that has been obtained directly from a subject, and the sample has been processed by removing one or more components of and/or by adding one or more agents to the clinical sample. For example, a sample may be filtered, purified, cleaned, decontaminated, centrifuged, or otherwise processed to isolate microbes or a microbial population within the sample from one or more components of the sample.

In any of the embodiments, methods, systems or modes described herein, the sample may be further processed by adding one or more processing reagents to the sample to degrade unwanted molecules present in the sample and/or dilute the sample for further processing. These processing reagents include, but are not limited to, surfactants and detergents, salts, cell lysing reagents, anticoagulants, degradative enzymes (e.g., proteases, lipases, nucleases, lipase, collagenase, cellulases, amylases, heparinases, and the like), and solvents, such as buffer solutions. Amount of the processing reagent to be added can depend on the particular sample to be analyzed, the time required for the sample analysis, identity of the microbe to be detected or the amount of microbe present in the sample to be analyzed.

It is not necessary, but if one or more reagents are to be added they can present in a mixture (e.g., in a solution, “processing buffer”) in the appropriate concentrations. Amount of the various components of the processing buffer can vary depending upon the sample, microbe to be detected, concentration of the microbe in the sample, or time limitation for analysis.

The processing buffer can be made in any suitable buffer solution known the skilled artisan. Such buffer solutions include, but are not limited to, TBS, PBS, BIS-TRIS, BIS-TRIS Propane, HEPES, HEPES Sodium Salt, MES, MES Sodium Salt, MOPS, MOPS Sodium Salt, Sodium Chloride, Ammonium acetate solution, Ammonium formate solution, Ammonium phosphate monobasic solution, Ammonium tartrate dibasic solution, BICINE buffer Solution, Bicarbonate buffer solution, Citrate Concentrated Solution, Formic acid solution, Imidazole buffer Solution, IVIES solution, Magnesium acetate solution, Magnesium formate solution, Potassium acetate solution, Potassium acetate solution, Potassium acetate solution, Potassium citrate tribasic solution, Potassium formate solution, Potassium phosphate dibasic solution, Potassium phosphate dibasic solution, Potassium sodium tartrate solution, Propionic acid solution, STE buffer solution, STET buffer solution, Sodium acetate solution, Sodium formate solution, Sodium phosphate dibasic solution, Sodium phosphate monobasic solution, Sodium tartrate dibasic solution, TNT buffer solution, TRIS Glycine buffer solution, TRIS acetate-EDTA buffer solution, Triethylammonium phosphate solution, Trimethylammonium acetate solution, Trimethylammonium phosphate solution, Tris-EDTA buffer solution, TRIZMA® Base, and TRIZMA® HCL. Alternatively, the processing buffer can be made in water.

After addition of the processing reagents, the sample can be incubated for a period of time, e.g., for at least 1, 2, 3, 4, 5, 10, 15, 30, 45, or 60 minutes. Such incubation can be at any appropriate temperature, e.g., about 16° C. to about 30° C., room-temperature (e.g., about 20° C. to about 25° C.), a cold temperature (e.g. about 0° C. to about 16° C.), or an elevated temperature (e.g., about 30° C. to about 95° C.). In some embodiments, the sample is incubated for about fifteen minutes at room temperature.

In any of the embodiments, methods, systems or modes described herein, the microbe may be a bacteria, a fungi, a virus, a parasite, protozoa, or a microbial spore. In some embodiments, the bacteria is from any one of the following phyla: Acidobacteria, Actinobacteria, Aquificae, Armatimonadetes, Bacteroidetes, Caldiserica, Chlamydiae, Chlorobi, Chloroflexi, Chrysiogenetes, Cyanobacteria, Deferribacteres, Deinococcus, Dictyoglomi, Elusimicrobia, Fibrobacteres, Firmicutes, Fusobacterial, Gemmatimonadetes, Lentisphaerae, Nitrospirae, Planctomycetes, Proteobacteria, Spirochaetes, Synergistetes, Tenericutes, Thermodesulfobacteria, Thermomicrobia, Thermotogae, or Verrucomicrobia, or mutants and derivatives of any of the microbial species, such as those produced by genetic and/or recombinant techniques.

In any of the embodiments, methods, systems or modes described herein, the bacteria may be a gram positive bacterium or a gram negative bacterium. In some embodiments, the bacterium is an aerobic bacterium or an anaerobic bacterium. In some embodiments, the bacterium is an autotrophic bacterium or a heterotrophic bacterium. In some embodiments, the bacterium is a mesophile, a neutrophile, an extremophile, an acidophile, an alkaliphile, a thermophile, a psychrophile, a halophile, or an osmophile.

In any of the embodiments, methods, systems or modes described herein, the bacterium may be an anthrax bacterium, an antibiotic resistant bacterium, a disease causing bacterium, a food poisoning bacterium, an infectious bacterium, Salmonella bacterium, Staphylococcus bacterium, Streptococcus bacterium, or tetanus bacterium. In some embodiments, the bacterium can be a mycobacteria, Clostridium tetani, Yersinia pestis, Bacillus anthraces, methicillin-resistant Staphylococcus aureus (MRSA), or Clostridium difficile. In some embodiments, the bacterium can be Mycobacterium tuberculosis. In some embodiments, the bacterium is Escherichia coli, Pseudomonas aeruginosa, Staphylococcus aureus, Staphylococcus epidermidis, Enterococcus faecalis, Klebsiella pneumoniae, Enterobacter cloacae, Acinetobacter baumanii, Serratia marcescens, or Enterococcus faecium.

In any of the embodiments, methods, systems or modes described herein, the microbe may be a protozoa causing diseases such as malaria, sleeping sickness, or toxoplasmosis; a fungi causing diseases such as ringworm, candidiasis or histoplasmosis. The term “microbe” or “microbes” can also encompass non-pathogenic microbes, e.g., a microbe used in industrial applications.

As used herein, the section headings are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein.

In this application, the use of the singular includes the plural unless specifically stated otherwise. Also, the use of “comprise”, “comprises”, “comprising”, “contain”, “contains”, “containing”, “include”, “includes”, and “including” are not intended to be limiting.

As used in this specification and claims, the singular forms “a,” “an” and “the” include plural references unless the content clearly dictates otherwise.

Although this invention has been disclosed in the context of certain embodiments and examples, those skilled in the art will understand that the present invention extends beyond the specifically disclosed embodiments to other alternative embodiments and/or uses of the invention and obvious modifications and equivalents thereof. In addition, while several variations of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. It should be understood that various features and aspects of the disclosed embodiments can be combined with, or substituted for, one another in order to form varying modes or embodiments of the disclosed invention. Thus, it is intended that the scope of the present invention herein disclosed should not be limited by the particular disclosed embodiments described above.

It should be understood, however, that this detailed description, while indicating preferred embodiments of the invention, is given by way of illustration only, since various changes and modifications within the spirit and scope of the invention will become apparent to those skilled in the art.

The terminology used in the description presented herein is not intended to be interpreted in any limited or restrictive manner. Rather, the terminology is simply being utilized in conjunction with a detailed description of embodiments of the systems, methods and related components. Furthermore, embodiments may comprise several novel features, no single one of which is solely responsible for its desirable attributes or is believed to be essential to practicing the inventions herein described.

What is claimed is:

1. A method of assessing microbial proliferation in a sample comprising:
a) providing a sample comprising microbes;
b) separating the sample comprising microbes into one or more portions of sample comprising microbes;
c) forming one or more populations of microdroplets encapsulating microbes from the sample, wherein the one or more populations of microdroplets are formed before or after separating the sample into one or more portions;
d) contacting the one or more portions of sample with an antimicrobial either before or after forming one or more populations of microdroplets, wherein each of the one or more portions of the sample is contacted with a different concentration of an antimicrobial; and
e) measuring microbial viability of microbes encapsulated within microdroplets;
thereby determining susceptibility of the microbes to the antimicrobial.

2. The method of claim 1, wherein said measuring microbial viability comprises obtaining a measure of microbial viability from a discrete subset of microdroplets from a first population of microdroplets from a first portion of the sample measured at a first time point, and obtaining a measure of microbial viability from a discrete subset of microdroplets from a second population of microdroplets from the first portion of the sample measured at a second time point.

3. The method of claim 2, wherein an average of the measure of microbial viability from a plurality of discrete subsets of microdroplets measured at the first time point is compared to an average of the measure of microbial viability from a plurality of discrete subsets of microdroplets measured at the second time point.

4. The method of claim 2, wherein said measuring microbial viability further comprises comparing the measure of microbial viability from a discrete subset of microdroplets measured at the first time point to the measure of microbial viability from a discrete subset of microdroplets measured at the second time point for a plurality of subsets of microdroplets measured at the first and second time points.

5. The method of any one of claims 2-4, wherein the measurements of microbial viability obtained at the first and second time points are not assigned to discrete subsets of microdroplets.

6. The method of any one of claims 2-5, wherein one or more discrete subsets of microdroplets from the first population are not in the second population, and one or more discrete subsets of microdroplets from the second population are not in the first population.

7. The method of any one of claims 2-6, wherein measuring further comprises obtaining a measure of microbial viability from a discrete subset of microdroplets in an additional population of microdroplets from the first portion of the sample measured at an additional time point.

8. The method of any one of claims 2-7, wherein the populations of microdroplets are incubated for a period of any one or more of 0 hr, 0.1 hr, 0.2 hr, 0.5 hr, 1 hr, 2 hr, 3 hr, 4 hr, 5 hr, 6 hr, 7 hr, 8 hr, 9 hr, 10 hr, 11 hr, 12 hr, 15 hr, 18 hr, 21 hr, or 24 prior to measuring microbial viability.

9. The method of any one of claims 2-8, wherein the microdroplets are formed within 1 second, 30 seconds, 1 minute, 15 minutes, 30 minutes, 1 hour, or 2 hours of contacting the one or more portions of samples with the antimicrobial.

10. The method of claim 1, wherein said measuring microbial viability further comprises obtaining a measure of microbial viability from discrete subsets of microdroplets in a first population of microdroplets from a first portion of the sample measured at a first time point, and obtaining a measure of microbial viability from discrete subsets of microdroplets in a second population of microdroplets from the first portion of the sample measured at a second time point, further comprising assigning measurements obtained at the first and second time points to discrete subsets of microdroplets, wherein at least some of the discrete subsets of microdroplets in the first population are the same discrete subsets of microdroplets in the second population such that the measurement of microbial viability obtained for a discrete subset of microdroplets at the first time point can be compared to the measurement of microbial viability obtained for that same discrete subset of microdroplets obtained at the second time point.

11. The method of claim 10, wherein said measuring microbial viability further comprises comparing the measurement of microbial viability obtained for a discrete subset of microdroplets at the first time point to the measurement of microbial viability obtained for that same discrete subset of microdroplets obtained at the second time point.

12. The method of claim 10, wherein at least one discrete subset of microdroplets in the first population is not in the second population, and at least one discrete subset of microdroplets in the second population is not in the first population.

13. The method of any one of claims 10-12, wherein measuring further comprises obtaining a measure of microbial viability from a discrete subset of microdroplets in an additional population of microdroplets measured at an additional time point.

14. The method of any one of claims 10-13, wherein the populations of microdroplets are incubated for a period of any one or more of 0 hr, 0.1 hr, 0.2 hr, 0.5 hr, 1 hr, 2 hr, 3 hr, 4 hr, 5 hr, 6 hr, 7 hr, 8 hr, 9 hr, 10 hr, 11 hr, 12 hr, 15 hr, 18 hr, 21 hr, or 24 hr prior to measuring microbial viability.

15. The method of any one of claims 10-14, wherein the microdroplets are formed within 1 second, 30 seconds, 1 minute, 15 minutes, 30 minutes, 1 hour, or 2 hours of contacting the one or more portions of samples with the antimicrobial.

16. The method of claim 1, wherein measuring microbial viability comprises obtaining a measure of microbial viability from a discrete subset of microdroplets in a first population of microdroplets from a first portion of the sample measured at a first time point, and obtaining a measure of microbial viability from a discrete subset of microdroplets in a second population of microdroplets from the first portion of the sample measured at a second time point, wherein the measure of microbial viability is whether an indicator of microbial viability exceeds a preset threshold.

17. The method of claim 16, wherein a composite of the measure of microbial viability from a plurality of discrete subsets of microdroplets measured at the first time point is compared to a composite of the measure of microbial viability from a plurality of discrete subsets of microdroplets measured at the second time point.

18. The method of claim 17, wherein the composite of the measure of microbial viability is the percentage of the plurality of discrete subsets of microdroplets measured at a time point that exceeds the threshold.

19. The method of claim 16, wherein said measuring microbial viability further comprises comparing the measure of microbial viability from a discrete subset of microdroplets obtained at the first time point to the measure of microbial viability from a discrete subset of microdroplets obtained at the second time point for a plurality of subsets of microdroplets measured at the first and second time points.

20. The method of any one of claims 16-19, wherein the measurements of microbial viability obtained at the first and second time points are not assigned to discrete subsets of microdroplets.

21. The method of claim 16, wherein said measuring microbial viability further comprises comparing the measure of microbial viability obtained for a discrete subset of microdroplets at the first time point to the measure of microbial viability obtained for that same discrete subset of microdroplets obtained at the second time point, for a plurality of subsets of microdroplets.

22. The method of any one of claims 16 to 21, wherein one or more discrete subsets of microdroplets from the first population are not in the second population, and one or more discrete subsets of microdroplets from the second population are not in the first population.

23. The method of any one of claims 16-22, wherein the preset threshold is exceeded when an indicator reaches a determined measure of microbial viability.

24. The method of any one of claims 16-23, wherein measuring further comprises obtaining a measure of microbial viability from a discrete subset of microdroplets in an additional population of microdroplets measured at an additional time point if an indicator of microbial viability exceeds the preset threshold.

25. The method of any one of claims 16-22, wherein the population of microdroplets are incubated for a period of any one or more of 0 hr, 0.1 hr, 0.2 hr, 0.5 hr, 1 hr, 2 hr, 3 hr, 4 hr, 5 hr, 6 hr, 7 hr, 8 hr, 9 hr, 10 hr, 11 hr, 12 hr, 15 hr, 18 hr, 21 hr, or 24 hr prior to measuring microbial viability.

26. The method of any one of claims 16-23, wherein the microdroplets are formed within 1 second, 30 seconds, 1 minute, 15 minutes, 30 minutes, 1 hour, or 2 hours of contacting the one or more portions of samples with the antimicrobial.

27. The method of claim 1, further comprising incubating the one or more portions of samples contacted with antimicrobial for different periods of time prior to forming the one or more populations of microdroplets, whereby microdroplets are formed from each of the one or more portions of samples at different time points.

28. The method of claim 27, wherein the one or more portions of samples are incubated for a time period sufficient to monitor microbial viability.

29. The method of claim 28, wherein the one or more portions of samples are incubated for a time period sufficient to allow microbial quorum sensing.

30. The method of any one of claims 27-29, wherein the one or more portions of samples are incubated over a period of any one or more of 0 hr, 0.1 hr, 0.2 hr, 0.5 hr, 1 hr, 2 hr, 3 hr, 4 hr, 5 hr, 6 hr, 7 hr, 8 hr, 9 hr, 10 hr, 11 hr, 12 hr, 15 hr, 18 hr, 21 hr, or 24 hr prior to forming microdroplets.

31. The method of any one of claims 2-30, wherein susceptibility of a microorganism to an antibiotic is determined by measuring viability of microorganisms in the presence of difference concentrations of an antibiotic.

32. The method of any one of claims 2-31, wherein measuring microbial viability in droplets is performed using a technology that affects viability of the microorganism, including determination of bacterial concentration by genetic analysis, including qPCR or fluorescence in-situ hybridization (FISH) after bacterial lysis.

33. The method of any one of claims 2-31, wherein measuring microbial viability in droplets is performed using a technology that does not affect viability of the microorganism, including measurement of solution turbidity, pH or fluorescence of a metabolically active dye.

34. The method of any one of claims 27-33, wherein said measuring microbial viability comprises obtaining a measure of microbial viability from a discrete subset of microdroplets from a first population of microdroplets from a first portion of the sample measured at a first time point, and obtaining a measure of microbial viability from a discrete subset of microdroplets from a second population of microdroplets from the first portion of the sample measured at a second time point.

35. The method of claim 34, wherein the measure of microbial viability is whether an indicator of microbial viability exceeds a preset threshold.

36. The method of any one of claims 2-35, wherein the individual subset of microdroplets comprises one or more microdroplets.

37. The method of any one of claims 1-36, wherein the one or more portions of samples are cultured in a culture medium.

38. The method of claim 37, wherein the culture medium is added before formation of microdroplets or during formation of microdroplets.

39. The method of any one of claims 1-38, further comprising immobilizing the one or more populations of microdroplets encapsulating microbes on an indexed array.

40. The method of any one of claims 1-39, further comprising flowing the one or more populations of microdroplets encapsulating microbes through a high throughput microdroplet reader.

41. The method of any one of claims 1-40, wherein the different concentration of antimicrobial spans a desired clinical range in the range of 0.002 mg/L to 500 mg/L.

42. The method of any one of claims 1-41, wherein measuring microbial viability comprises measuring a fluorescence signal of a label.

43. The method of claim 42, wherein the fluorescence is measured using a fluorescence reader.

44. The method of any one of claims 42-43, wherein microbial viability is determined by measuring absorbance or electrochemical properties of a viability indicator dye.

45. The method claim 44, wherein the viability indicator dye comprises resazurin, formazan, or analogues or salts thereof.

46. The method of any one of claims 1-41, wherein microbial viability is determined by measuring absorbance or electrochemical properties of a viability indicator, or by measuring pH or turbidity.

47. The method of any one of claims 1-46, wherein an average number of microbes per microdroplet is less than 2.

48. The method of any one of claims 1-47, wherein an average number of microbes per microdroplet is less than 1.

49. The method of any one of claims 1-48, wherein the microbe is a bacteria.

50. The method of claim 49, wherein the bacteria is E. coli, P. aeruginosa, S. aureus, S. epidermidis, E. faecalis, K. pneumoniae, E. cloacae, A. baumanii, S. marcescens, or E. faecium.

51. The method of any one of claims 1-50, wherein the microbes are bacteria and wherein the antimicrobial is an antibiotic.

52. The method of claim 51, wherein the antibiotic is an aminocoumarin, an aminoglycoside, an ansamycin, a carbacephem, a carbapenem, a cephalosporin, a glycopeptide, a lincosamide, a lipopeptide, a macrolide, a monobactam, a nitrofuran, a penicillin, a polypeptide, a quinolone, a streptogramin, a sulfonamide, or a tetracycline, or a combination thereof.

53. The method of any one of claims 51-52, wherein the antibiotic is ampicillin.

54. The method of any one of claims 1-53, wherein the microdroplet comprises an oil phase and a surfactant phase.

55. The method of any one of claims 1-54, wherein the microdroplets are formulated by microfluidic channels, agitation, electric forces, or membrane filtration.

56. The method of any one of claims 1-55, wherein the one or more populations of microdroplets are formulated as stable water-in-oil emulsions.

57. The method of any one of claims 1-56, wherein the determining susceptibility of the microbes to the antimicrobial is completed more quickly than when no microdroplets are formed.

58. The method of any one of claims 1-57, wherein determining susceptibility of the microbes to the antimicrobial is completed in a time within a range of 3-24 hours, 3-20 hours, 3-15 hours, 3-8 hours, 5-20 hours, 5-15 hours, or 5-8 hours.

59. The method of any one of claims 1-58, wherein determining susceptibility of the microbes to the antimicrobial is completed in not more than 24 hours; in not more than 15 hours; in not more than 12 hours; in not more than 10 hours; in not more than 8 hours; in not more than 5 hours; in not more than 3 hours.

60. The method of any one of claims 1-59, wherein the sample is whole blood, positive blood culture, peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen (including prostatic fluid), Cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates or other lavage fluids, blastocoel cavity, umbilical cord blood, or maternal circulation.
